(19) United States
(12) Patent Application Publication
(10) Pub. No.: US 2010/0292088 A1
(43) Pub. Date: Nov. 18, 2010

(54) METHODS AND COMPOSITIONS FOR DETECTING GENETIC MARKERS ASSOCIATED WITH PRIMARY CILIARY DYSKINESIA

(75) Inventors: Maimoona Banoo Abdul Hamid Zariwala, Chapel Hill, NC (US); Michael R. Knowles, Chapel Hill, NC (US)

Correspondence Address: MYERS BIGEL SIBLEY & SAJOVEC, PO BOX 37428, RALEIGH, NC, 27627, US

(73) Assignee: The University of North Carolina at Chapel Hill

(21) Appl. No.: 12/780,220

(22) Filed: May 14, 2010

Related U.S. Application Data
(60) Provisional application No. 61/178,775, filed on May 15, 2009.

(51) Int. Cl. C40B 30/02; G01N 33/50; C12Q 1/68; G01N 23/00; G01N 30/00

(52) U.S. Cl. 506/8; 436/94; 435/6; 250/307; 73/61.52

(57) ABSTRACT

The present invention provides methods and compositions for detecting mutations in a DNAH11 gene of a subject to diagnose primary ciliary dyskinesia (PCD) in the subject and/or to identify a subject as having an increased risk of having PCD and/or to identify a carrier of a PCD mutation.

### STATEMENT OF PRIORITY

This application claims the benefit, under 35. U.S.C. §119(e), of U.S. Provisional Application Ser. No. 61/178,775, filed May 15, 2009, the entire contents of which are incorporated by reference herein.

### STATEMENT OF GOVERNMENT SUPPORT

Aspects of the present invention were made with the support of funding under federal grant numbers RR00046, CTSA UL1RR025747, 1 RO1 HL071798, 5 U54 RR19480 NO1-HV-48194R99 from the National Institutes of Health. The United States Government has certain rights to this invention.

### FIELD OF THE INVENTION

The present invention provides methods and compositions directed to identification of genetic markers associated with primary ciliary dyskinesia (PCD).

### BACKGROUND OF THE INVENTION

Primary ciliary dyskinesia (PCD) is usually an autosomal recessive trait reflecting abnormalities in the structure and function of cilia of the respiratory tract and flagella of the sperm. It is a rare genetic disorder, with an incidence of approximately 1 in 16,000, which corresponds to a carrier rate of approximately 1 in 63. It is estimated that there are 12-17,000 patients in the USA affected with PCD. Clinically, PCD is associated with recurrent sinusitis, middle ear disease (otitis media), pneumonia, bronchitis, and in most cases patients eventually develop end-stage bronchiectasis and require lung transplantation. It also causes infertility in males and reduced fertility in females. Approximately 50% of patients with PCD present with situs inversus totalis (total reversal of all internal visceral organs), termed Kartagener syndrome (KS), and at least 6% have heterotaxy (abnormal placement of organs due to failure to establish the normal left-right patterning during embryonic development.). It is a genetically heterogeneous disorder and mutations in multiple genes on various chromosomes can cause PCD (locus heterogeneity) or multiple mutations within a gene can cause PCD (allelic heterogeneity). But in any given PCD patient, two mutations (biallelic) each inherited from a parent from one PCD-causing gene are sufficient to cause the disease. Diagnosis of PCD is made on the basis of clinical criteria, together with the documentation of the presence of defective ciliary ultrastructure using electron microscopy. The majority of the PCD patients (80-90%) have documented ciliary outer (ODA) and inner (IDA) dynein arms abnormalities.

Mutations in two ciliary outer dynein arm genes, DNAI1 and DNAH5, have been shown to account for 10% and 28% of cases in PCD, respectively. A clinical genetic test for PCD is available that analyzes a limited number of mutations but is diagnostic only in a small fraction of patients. Mutations in other ciliary genes have also been revealed, but in a very small number (1-5 family) of PCD families. Levels of nasal nitric oxide (NO) are low in PCD patients, which aids in the diagnosis if cystic fibrosis is ruled out, but it is only used as an adjunct screening test because there is no Food and Drug Administration (FDA) approved device for detection of nasal NO. Diagnosis of PCD in patients who present with compatible clinical phenotype and low nasal NO without the documentation of the ultrastructural defects is difficult, because ultrastructural analysis is the gold standard for the diagnosis.

The present invention overcomes previous shortcomings in the art by providing methods and compositions for diagnosing PCD in a subject by detecting PCD mutations in the DNAH11 gene of the subject.

### SUMMARY OF THE INVENTION

In one aspect, the present invention provides a method of diagnosing primary ciliary dyskinesia (PCD) in a subject, comprising detecting in the subject at least two mutations of this invention (i.e., PCD mutations) in the DNAH11 gene of the subject.

An additional aspect of this invention is a method of confirming a diagnosis of PCD in a subject, comprising detecting in the subject at least two mutations of this invention (i.e., PCD mutations) in the DNAH11 gene of the subject.

Additionally provided herein is a method of identifying a subject as having an increased likelihood of having PCD, comprising detecting at least two mutations of this invention (i.e., PCD mutations) in the DNAH11 gene of the subject.

Furthermore, the present invention provides a method of identifying a carrier of a PCD mutation of this invention and/or of identifying a subject having an increased likelihood of having PCD, comprising detecting in the subject at least one mutation of this invention (i.e., a PCD mutation) in the DNAH11 gene of the subject.

As an additional aspect, the present invention provides a kit comprising reagents to detect one or more mutation of this invention (i.e., a PCD mutation) in a DNAH11 gene.

In an additional embodiment, the present invention provides a computer-assisted method of identifying a proposed treatment for PCD as an effective and/or appropriate treatment for a subject carrying a PCD mutation, comprising the steps of: (a) storing a database of biological data for a plurality of subjects, the biological data that is being stored including for each of said plurality of subjects: (i) a treatment type, (ii) at least one PCD mutation, and (iii) at least one disease progression measure for PCD from which treatment efficacy can be determined; and then (b) querying the database to determine the dependence on said PCD mutation of the effectiveness of a treatment type in treating PCD, thereby identifying a proposed treatment as an effective and/or appropriate treatment for a subject carrying a PCD mutation.

In any or all of the embodiments described above, the mutation of this invention (i.e., a PCD mutation) can be
1) 350A>T (E117V);
2) IVS13−1G>C (c.2275−1G>C); (Y759_E889del)
3) 2569C>T (R857X);
4) 3901G>T (E1301X);
5) IVS23+5G>T (c. 4254+5G>T) (E1366_G1418del);
6) 4333C>T (R1445X);
7) 4438C>T (R1480X);
8) 4516_{—}4517delCT (L1506fsX10);
9) IVS26−1G>A (c. 4726−1G>A) (E1576AfsX4);
10) IVS33+1G>A (c. 5778+1G>A) (V1821TfsX7);
11) 5815G>A (G1939R);
12) 6244C>T (R2082X);
13) 7148T>C (L2383P);
14) IVS44+1G>A (T2379_Q2422del)
15) 7914G>C (W2604X splice)
16) 9113_{—}9116delAAGA (K3038TfsX13);
17) 9764T>C (L3255S);
18) 10324C>T (Q3442X);
19) 11663G>A (R3888H);
20) 11804C>T (P3935L));
21) 11929G>T (E3977X);
22) 12064G>C (A4022P);
23) 12697C>T (Q4233X);
24) 12980T>C (L4327S);
25) 13061T>A (L4354H);
26) 13065_{—}13067delCCT (4356delL);
27) 13075C>T (R4359X);
28) 13213delC (R4405AfsX1);
29) 13333_{—}13334insACCA (I4445NfsX3);
30) 13504_{—}13505insGAAGA (T4502RfsX14);
31) 13373C>T (P4458L); or
32) any combination of (1)-(31) above.

Also provided herein is a computer-assisted method of identifying a proposed therapy and/or treatment for PCD as an effective and/or appropriate therapy and/or treatment for a subject that has PCD, comprising the steps of: (a) storing a database of biological data for a plurality of subjects, the biological data that is being stored including for each of said plurality of subjects: (i) therapy and/or treatment type, (ii) at least one PCD mutation, and (iii) at least one disease progression measure and/or symptom for PCD from which treatment and/or therapy efficacy can be determined; and then (b) querying the database to determine the dependence on said PCD mutation(s) of the effectiveness of a treatment and/or therapy type in treating and/or managing PCD, thereby identifying a proposed treatment and/or therapy as an effective and/or appropriate treatment and/or therapy for a subject with PCD.

These aspects and embodiments of the present invention are explained in greater detail below.

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

The present invention is based on the unexpected discovery that particular mutations in the DNAH11 gene are associated with PCD. Thus, in one aspect, the present invention provides a method of diagnosing primary ciliary dyskinesia (PCD) in a subject, comprising detecting the presence or absence of at least two PCD mutations in the DNAH11 gene of the subject and then determining that the subject is diagnosed with PCD due to the presence or absence of the at least two PCD mutations.

An additional aspect of this invention is a method of confirming a diagnosis of PCD in a subject, comprising detecting the presence or absence of at least two PCD mutations in the DNAH11 gene of the subject and then confirming the diagnosis of PCD in the subject due to the presence or absence of the at least two PCT mutations.

Additionally provided herein is a method of identifying a subject as having an increased likelihood of having PCD, comprising detecting the presence or absence of at least two PCD mutations in the DNAH11 gene of the subject and then identifying the subject as having an increased likelihood of having PCD due to the presence or absence of the at least two PCD mutations.

Furthermore, the present invention provides a method of identifying a carrier of a PCD mutation of this invention, comprising detecting the presence or absence of at least one PCD mutation in the DNAH11 gene of a subject and then identifying the subject as a carrier of a PCD mutation due to the presence or absence of the at least one PCD mutation.

Also provided herein is a method of identifying a subject as having an increased likelihood of having PCD, comprising detecting the presence or absence of at least one PCD mutation in the DNAH11 gene of the subject and then identifying the subject as having an increased likelihood of having PCD due to the presence of absence of the at least one PCD mutation.

As used herein, a “PCD mutation” is any of the following mutations, singly or in any combination. The first description is of the nucleotide sequence alteration and the description in parentheses is of the resulting alteration at the amino acid sequence level (e.g., 350A>T identifies an A to T mutation at nucleotide 350 in the DNAH11 gene and E117V identifies an E to V mutation in the amino acid sequence of the DNAH11 gene product) Also, X identifies a mutation site where a base substitution leads to a stop codon (i.e., TAA, TGA or TAG), which results in a stop signal for the developing amino acid chain and truncation of the protein.
1) 350A>T (E117V);
2) IVS13−1G>C (c.2275−1G>C); (Y759_E889del)
3) 2569C>T (R857X);
4) 3901G>T (E1301X);
5) IVS23+5G>T (E1366_G1418del);
6) 4333C>T (R1445X);
7) 4438C>T (R1480X);
8) 4516_{—}4517delCT (L1506fsX10);
9) IVS26−1G>A (E1576AfsX4);
10) IVS33+1G>A (V1821TfsX7) (identified as IVS34+1G>A in Ensembl sequence number ENSG00000105877; intron sequence shown herein);
11) 5815G>A (G1939R);
12) 6244C>T (R2082X);
13) 7148T>C (L2383P);
14) IVS44+1G>A (identified as IVS45+1G>A in Ensemble sequence number ENSG00000105877; intron sequence shown herein); (T2379_Q2422del)
15) 7914G>C (W2604X splice)
16) 9113_{—}9116delAAGA (K3038TfsX13);
17) 9764T>C (L3255S);
18) 10324C>T (Q3442X);
19) 11663G>A (R3888H);
20) 11804C>T (P3935L));
21) 11929G>T (E3977X);
22) 12064G>C (A4022P);
23) 12697C>T (Q4233X);
24) 12980T>C (L4327S);
25) 13061T>A (L4354H);
26) 13065_{—}13067delCCT (4356delL);
27) 13075C>T (R4359X);
28) 13213delC (R4405AfsX1);
29) 13333_{—}13334insACCA (I4445NfsX3);
30) 13504_{—}13505insGAAGA (T4502RfsX14);
31) 13373C>T (P4458L); or
32) any combination of (1)-(31) above.

Numbering of the nucleotides of the DNAH11 nucleotide sequence and of the amino acid sequence of the DNAH11 gene product for mutations 1, 3, 4, 6, 7, 8, 11, 12, 13 and 15-31 is based on the reference DNAH11 cDNA and amino acid sequence provided herein (Table 4), which is an updated sequence that corrects errors identified by the inventors in the previously disclosed DNAH11 sequence having Ensembl number ENSG00000105877. A description of the errors identified and the change in numbering of the nucleotides and corresponding amino acids is provided in Table 3. Numbering of the nucleotides of the DNAH11 gene for mutations 2, 5, 9, 10 and 14 (in the intron sequences) is based on the reference nucleotide sequence identified in the Ensembl database under number ENSG00000105877. The nucleotide sequence of the intron in which each of these intron mutations is located is provided herein.

The present invention encompasses a single PCD mutation, as well as any combination of two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31) of the PCD mutations of this invention. Nonlimiting examples of combinations of two PCD mutations of this invention include mutations (8) and (14), mutations (12) and (21), mutations (10) and (25), mutations (2) and (28), mutations (11) and (31), mutations (26) and (27), mutations (4) and (20), mutations (22) and (30), mutations (15) and (29), mutations (1) and (13), mutations (23) and (24), and mutations (5) and (9).

The reference sequences and mutation numbering described herein are based on the human DNAH11 gene; however the present invention encompasses homologues of the human DNAH11 gene from other species, with mutations in said homologues corresponding to the mutations of the human DNAH11 gene as would be readily identifiable to one of ordinary skill in the art.

A subject of this invention is any animal, male or female, that is susceptible to PCD as defined herein and can include, for example, humans, as well as animal models of PCD (e.g., rats, mice, dogs, etc. See, e.g., Leigh et al. “Clinical and genetic aspects of primary ciliary dyskinesia/Katgegener syndrome” Genetics In Medicine, volume 11, no. 7, online publication April, 2009, the entire contents of which are incorporated by reference herein). In some aspects of this invention, the subject can be a Caucasian (e.g., white; European-American; Hispanic) human and in other aspects the subject can be a human of black African ancestry (e.g., black; African American; African-European; African-Caribbean, etc.). In yet other aspects the subject can be Asian or Mid-eastern.

The subject of this invention can be a subject identified to have normal dynein arm ultrastructure as analyzed by transmission electron microscopy (TEM) [e.g., dynein arm ultrastructure that does not show PCD-associated defects (e.g., shortening and/or absence of dynein arms (inner, outer or both) and/or absence or disruption of the central apparatus (central microtubule pair and/or radial spokes) (MacCormick et al. 2002 “Optimal biopsy techniques n the diagnosis of primary ciliary dyskinesia” J. Otolaryngol. 31:13-17; Chilvers et al. 2003 “Ciliary beat pattern is associated with specific ultrastructural defects in primary ciliary dyskinesia” J. Allergy Clin. Immunol. 112:518-524)]. The subject of this invention can also be a subject with abnormal dynein arm ultrastructure (e.g., characteristic of PCD).

Additionally in some embodiments, a subject of this invention can have a diagnosis of PCD and in other embodiments, a subject of this invention does not have a diagnosis of PCD. A subject of this invention can also be a subject having symptoms of PCD but without a diagnosis of PCD.

In further aspects of this invention, the subject has a family history of PCD (e.g., having at least one first degree relative diagnosed with PCD) and in some embodiments, the subject does not have a family history of PCD. The subject can further be a subject with a relative that has a diagnosis of PCD or has symptoms of PCD without a diagnosis of PCD. For such subjects, a diagnosis of PCD can be confirmed by carrying out the methods of this invention. A carrier of a PCD mutation of this invention can also be identified by carrying out the methods of this invention.

Detection of the PCD mutations of this invention in the DNAH11 gene of a subject can provide a diagnosis of PCD in a subject, as well as confirmation of a diagnosis of PCD in a subject (e.g., a subject suspected to have PCD). This is based on the inventors' identification of the majority of these mutations as truncating mutations and as clearly identifiable mutations. Specifically, for the splice mutations, in vitro assays were done to check the effect on the transcripts whenever RNA samples were available from a subject. In the absence of the availability of RNA, in silico splicing prediction programs were used to check if the mutation is predicted to cause the splicing defects. In addition, the conservation of the splicing canonical sites was considered. As for the missense mutations, population studies were done to check that the missense mutation was rare in the control group. Also, for the missense mutations, evolutionary conservation across the species was used to predict if the change is intolerant. Furthermore, a majority of the subjects had two mutations identified and inherited from each parent (when DNA was available from the parents), which is consistent with the autosomal recessive mode of inheritance of the disorder. In some cases, only one mutation was identified, but that may be because full exon and intron/exon junction sequencing cannot identify 100% of the mutations because some mutations can reside in regions not covered by sequencing.

The present invention further provides a kit comprising reagents to detect one or more PCD mutation of this invention in a DNAH11 gene in a nucleic acid sample from a subject. Such a kit can comprise primers, probes, primer/probe sets, reagents, buffers, etc., as would be known in the art, for the detection of the PDC mutations of this invention in a nucleic acid sample from a subject. For example, a primer or probe can comprise a contiguous nucleotide sequence that is complementary to a region comprising one or more than one PCD mutation of this invention. In particular embodiments, a kit of this invention can comprise primers and probes that allow for the specific detection of the PCD mutations of this invention. Such a kit can further comprise blocking probes, labeling reagents, blocking agents, restriction enzymes, antibodies, sampling devices, positive and negative controls, etc., as would be well known to those of skill in the art.

### Definitions

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, the term “primary ciliary dyskinesia” or “PCD” describes an autosomal recessive, genetically heterogeneous disorder characterized by oto-sino-pulmonary disease due to abnormal structure and function of cilia. Most PCD patients (˜90%) have ultrastructural defects of cilia involving the outer dynein arm (ODA), or inner dynein arm (IDA) or both arms (DA). Disease-causing mutations in DNAI1 and DNAH5 genes (encoding ODA proteins) account for 38% of PCD. There are many patients with clinical manifestations of PCD and normal DA ultrastructure and low nasal nitric oxide (NO) levels and definitive diagnosis in patients without ultrastructural defects is difficult.

PCD is characterized by clinical manifestations and/or symptoms that can include abnormal ciliary structure leading to characteristic defects, abnormal ciliary function, impaired mucociliary clearance, neonatal respiratory distress in full-term neonates, chronic productive cough, chronic middle ear, sinus and lung disease, immotile sperm (causing infertility in males in some cases) and reduced fertility in females in some cases. Approximately 50% of PCD patients have situs inversus totalis, termed Kartagener syndrome and at least 6% of PCD patients have heterotaxy.

Diagnosis of PCD currently requires the presence of the characteristic clinical phenotype and either specific ultrastructural defects identified by TEM in biopsy samples of the respiratory epithelium or evidence of abnormal ciliary function.

Management of PCD includes treatment of various manifestations, including aggressive measures to enhance clearance of mucus (chest percussion and postural drainage, oscillatory vest, breathing maneuver to facilitate clearance of distal airways) and antibiotic therapy for bacterial infections of the airways (bronchitis, sinusitis and otitis media); consideration of lobectomy for localized bronchiectasis; lung transplantation for end-stage lung disease; sinus surgery for extensive sinus infections; consideration of PE tube replacement for chronic otitis media; speech therapy and hearing aids as needed; surgical intervention as needed for congenital heart disease; and intracytoplasmic sperm injections (ICSI) or artificial insemination by donor sperm for male infertility. Secondary complications are managed by prevention of respiratory infection through routine immunization [see also Zariwala et al. Jan. 24, 2007 “Primary Ciliary Dyskinesia” in Gene Reviews (online publication from the University of Washington, Seattle Wash. (www.ncbi.nlm.nih.gov/bookshelf/br.fcgi?book=gene&part=pcd) and Leigh et al. “Primary ciliary dyskinesia: Improving the diagnostic approach” Curr. Opin. Pediatr. (Epub ahead of print Mar. 18, 2009); the entire contents of each of which are incorporated by reference herein].

The terms “increased risk” or “increased likelihood” as used herein defines the level of risk or the likelihood that a subject has of having PCD, as compared to a control subject that does not have the PCD mutation(s) of this invention in the control subject's DNAH11 gene.

A sample of this invention can be any sample containing nucleic acid of a subject, as would be well known to one of ordinary skill in the art. Nonlimiting examples of a sample of this invention include a cell, a body fluid, a tissue, a washing, a swabbing, etc., as would be well known in the art.

As used herein, “nucleic acid” encompasses both RNA and DNA, including cDNA, genomic DNA, mRNA, synthetic (e.g., chemically synthesized) DNA and chimeras, fusions and/or hybrids of RNA and DNA. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides, etc.). Such oligonucleotides can be used, for example, to prepare nucleic acids that have altered base-pairing abilities or increased resistance to nucleases.

An “isolated nucleic acid” is a nucleotide sequence that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid or polypeptide that is substantially free of cellular material, viral material, and/or culture medium (e.g., when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

The term “oligonucleotide” refers to a nucleic acid sequence of at least about six nucleotides to about 100 nucleotides, for example, about 15 to about 30 nucleotides, or about 20 to about 25 nucleotides, which can be used, for example, as a primer in a PCR amplification and/or as a probe in a hybridization assay or in a microarray. Oligonucleotides of this invention can be natural or synthetic, e.g., DNA, RNA, PNA, LNA, modified backbones, etc., as are well known in the art.

The present invention further provides fragments of the nucleic acids of this invention, which can be used, for example, as primers and/or probes. Such fragments or oligonucleotides can be detectably labeled or modified, for example, to include and/or incorporate a restriction enzyme cleavage site when employed as a primer in an amplification (e.g., PCR) assay.

The detection of a PCD mutation or multiple PCD mutations of this invention can be carried out according to various protocols standard in the art and as described herein for analyzing nucleic acid samples and nucleotide sequences, as well as identifying specific nucleotides and/or alterations (e.g., deletions, insertions, substitutions) in a nucleotide sequence.

For example, nucleic acid can be obtained from any suitable sample from the subject that will contain nucleic acid and the nucleic acid can then be prepared and analyzed according to well-established protocols for the presence of genetic markers according to the methods of this invention. In some embodiments, analysis of the nucleic acid can be carried by amplification of the region of interest according to amplification protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA), etc.). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, and/or by electrophoresis. Thus, the present invention further provides oligonucleotides for use as primers and/or probes for detecting and/or identifying genetic markers according to the methods of this invention.

Additional methods for detecting the mutations of this invention include but are not limited to sequencing, high performance liquid chromatography (HPLC), restriction enzyme analysis (e.g., restriction fragment length polymorphism or RFLP), hybridization, etc., all of which are well known protocols for analyzing a nucleotide sequence and detecting mutations. The methods of this invention can be carried out by using any assay or procedure that can interrogate a nucleic acid sequence.

The mutations of this invention are or can be correlated with (i.e., identified to be statistically associated with) PCD as described herein according to methods well known in the art and as disclosed in the Examples provided herein for statistically correlating genetic markers with various phenotypic traits, including disease states and pathological conditions as well as determining levels of risk or likelihood associated with developing or having a particular phenotype, such as a disease, disorder or pathological condition. In general, identifying such correlation involves conducting analyses that establish a statistically significant association and/or a statistically significant correlation between the presence of a genetic marker (e.g., mutation) or a combination of markers and the phenotypic trait in a population of subjects and controls (e.g., ethnically matched controls; gender matched controls, etc.). The correlation can involve one or more than one genetic marker of this invention (e.g., two, three, four, five, or more) in any combination. An analysis that identifies a statistical association (e.g., a significant association) between the marker or combination of markers and the phenotype establishes a correlation between the presence of the marker or combination of markers in a population of subjects and the particular phenotype being analyzed. A level of risk or likelihood (e.g., increased or decreased) can then be determined for an individual on the basis of such population-based analyses.

The present invention further provides a method of identifying an effective and/or appropriate (i.e., for a given subject's particular condition or status) treatment regimen for a subject with PCD, comprising detecting one or more of the PCD mutations of this invention in the subject, wherein the one or more PCD mutations are further statistically correlated with an effective and/or appropriate treatment regimen for PCD according to protocols as described herein and as are well known in the art.

Also provided is a method of identifying an effective and/or appropriate treatment regimen for a subject with PCD, comprising: a) correlating the presence of one or more PCD mutations of this invention in a test subject or population of test subjects with PCD for whom an effective and/or appropriate treatment regimen has been identified; and b) detecting the one or more PCD mutations of step (a) in the subject, thereby identifying an effective and/or appropriate treatment regimen for the subject.

Further provided is a method of correlating a PCD mutation of this invention with an effective and/or appropriate treatment regimen for PCD, comprising: a) detecting in a subject or a population of subjects with PCD and for whom an effective and/or appropriate treatment regimen has been identified, the presence of one or more PCD mutations of this invention; and b) correlating the presence of the one or more PCD mutations of step (a) with an effective treatment regimen for PCD.

Examples of treatment/management regimens for PCD are well known in the art.

Subjects who respond well to particular treatment protocols can be analyzed for specific genetic markers and a correlation can be established according to the methods provided herein. Alternatively, subjects who respond poorly to a particular treatment regimen can also be analyzed for particular genetic markers correlated with the poor response. Then, a subject who is a candidate for treatment for PCD can be assessed for the presence of the appropriate genetic markers and the most effective and/or appropriate treatment regimen can be provided.

In some embodiments, the methods of correlating genetic markers with treatment regimens of this invention can be carried out using a computer database. Thus, in some embodiments, the present invention provides a computer-assisted method of identifying a proposed therapy and/or treatment for PCD as an effective and/or appropriate therapy and/or treatment for a subject that has PCD, comprising the steps of: (a) storing a database of biological data for a plurality of subjects, the biological data that is being stored including for each of said plurality of subjects: (i) therapy and/or treatment type, (ii) at least one PCD mutation, and (iii) at least one disease progression measure and/or symptom for PCD from which treatment and/or therapy efficacy can be determined; and then (b) querying the database to determine the dependence on said PCD mutation(s) of the effectiveness of a treatment and/or therapy type in treating and/or managing PCD, thereby identifying a proposed treatment and/or therapy as an effective and/or appropriate treatment and/or therapy for a subject with PCD.

In one embodiment, treatment information for a subject is entered into the database (through any suitable means such as a window or text interface), genetic marker information for that subject is entered into the database, and disease progression responsiveness to treatment information is entered into the database. These steps are then repeated until the desired number of subjects has been entered into the database. The database can then be queried to determine whether a particular treatment is effective for subjects carrying a particular marker or combination of markers, not effective for subjects carrying a particular marker or combination of markers, etc. Such querying can be carried out prospectively or retrospectively on the database by any suitable means, but is generally done by statistical analysis in accordance with known techniques, as described herein.

The present invention is more particularly described in the following examples that are intended as illustrative only since numerous modifications and variations therein will be apparent to those skilled in the art.

### Examples

To test DNAH11 as a candidate gene for PCD, mutation analysis of the 82 coding exons and intron/exon junctions was carried out in 164 unrelated well-characterized PCD patients (n=59 had normal ciliary ultrastructure, n=74 had ODA defect, n=8 had central pair defect and n=23 ultrastructure not available). The majority of the patients were Caucasian; however two siblings (PCD918 and 919) of one family were of Pakistani origin and patient PO20 was of Turkish origin. Of the 59 patients with normal ultrastructure, 13 (22%) harbored biallelic DNAH11 mutations and 4 (7%) had only one mutation identified (Table 1). Two additional patients from whom ciliary ultrastructure was not available harbored biallelic mutations. The most pertinent finding was that mutations were exclusively identified in patients who had no ultrastructural ciliary defects. A total of 31 mutant alleles were noted (Table 2), of which nine were stop mutations (29%), six were frame-shift mutations (19%), seven were splice site mutations (23%) and nine were missense mutations (29%). Taken together, 22 (71%) were loss of function mutations. Mutations were seen in patients who had PCD based on their pulmonary disease, and nasal NO levels that were low. PCD is usually an autosomal recessive disorder and whenever possible it has been shown that biallelic mutations were inherited in trans (one from each parent). No gender bias or ethnic or racial bias was seen with respect to the mutations of the DNAH11 gene (e.g., with the same families, mutations in the DNAH11 gene were present in all affected siblings irrespective of gender). These results demonstrate that genetic analysis of the dynein gene can confirm PCD in the absence of ultrastructural defects. In addition nine nonsense mutations have been defined, which will be useful for therapy related to read-through of the premature termination codon.

The foregoing is illustrative of the present invention, and is not to be construed as limiting thereof. The invention is defined by the following claims, with equivalents of the claims to be included therein.

All publications, patent applications, patents, patent publications, all sequences identified by GenBank® database and/or SNP accession numbers, and other references cited herein are incorporated by reference in their entireties for the sequences and/or teachings relevant to the sentence and/or paragraph and/or claim in which the reference is presented.

TABLE 1
Table 1: PCD patients with mutations in DNAH11 gene

| Subject | | nNO | | | Neo | Otitis | Bronchi- | | Mutation |
|---|---|---|---|---|---|---|---|---|---|
| # | Sex | nl/min | Situs | DA defect | RDS | Media | ectasis | Sinusitis | Allele 1 |
| PCD106 | M | 14 | SS | normal DA | no | yes | no | yes | 4516_4517delCT |
| PCD108 | M | 20 | SI | normal DA | yes | yes | no | yes | 4516_4517delCT |
| PCD157 | F | 2.1 | SI | normal DA | yes | yes | no | yes | 6244C > T |
| PCD565 | M | 23.5 | SI | normal DA | yes | yes | no | yes | IVS33 + 1G > A |
| PCD623 | F | 9.7 | SS | normal DA | no | yes | yes | na | 4438C > T |
| PCD627 | F | na | SS | sib normal | na | na | na | na | 4438C > T |
| | | | | DA | | | | | |
| PCD761 | F | 24.5 | SA | normal DA | yes | yes | yes | yes | IVS13 − 1G > C |
| PCD812 | M | 9 | SI | no EMs | yes | yes | no | yes | 5815G > A |
| PCD918 | F | 19.4 | SS | normal DA | na | na | na | na | 13065_13067delCCT |
| PCD919 | M | 25.5 | SA | normal DA | na | yes | na | yes | 13065_13067delCCT |
| PCD974 | F | 40 | SS | normal DA | no | yes | yes | yes | 9764T > C |
| PCD998 | M | 70.4 | SS | normal DA | no | yes | yes | yes | 9113_9116delAAGA |
| PCD1022 | M | 12.5 | SS | normal DA | yes | yes | no | yes | 4333C > T |
| PCD1023 | M | 12.6 | SI | normal DA | yes | yes | no | yes | 4333C > T |
| PCD1033 | F | 34.8 | SA | normal DA | yes | yes | no | yes | 10324C > T |
| PCD1077 | F | 16.9 | SI | normal DA | yes | yes | no | yes | 3901G > T |
| PCD1126 | F | 16.2 | SS | normal DA | no | no | yes | yes | 12064G > C |
| PCD1174 | F | 32.1 | SS | normal DA | yes | yes | yes | yes | 2569C > T |
| OP-98 II1 | M | na | SI | normal DA | no | yes | yes | yes | 7914G > C |
| OP-98 II2 | M | na | SS | normal DA | na | yes | yes | yes | 7914G > C |
| OP-41 II1 | M | na | SI | normal DA | yes | yes | na | yes | 350A > T |
| OP-235 II1 | F | na | SS | normal DA | no | yes | yes | yes | 12697C > T |
| OP-235 II2 | F | na | SI | normal DA | yes | yes | yes | yes | 12697C > T |
| *OP-20 II1 | M | na | SI | no EMs | na | na | na | na | 11663G > A |
| OP-406 II1 | M | na | SI | normal DA | na | na | na | na | IVS23 + 5G > T |
| OP-406 II2 | F | na | SS | normal DA | yes | na | na | yes | IVS23 + 5G > T |

| | | | | | | **Were |
|---|---|---|---|---|---|---|
| Subject | Mutation | Location | Mutation | Mutation | Location | Mutations |
| # | Allele 1 | Allele 1 | Allele 2 | Allele 2 | Allele 2 | in trans |
| PCD106 | L1506SfsX10 | Ex 26 | IVS44 + 1G > A | T2379_Q2422del | Int 44 | yes |
| PCD108 | L1506SfsX10 | Ex 26 | IVS44 + 1G > A | T2379_Q2422del | Int 44 | yes |
| PCD157 | R2082X | Ex 37 | 11929G > T | E3977X | Ex 73 | yes |
| PCD565 | V1821TfsX7 | Int 33 | 13061T > A | L4354H | Ex 80 | yes |
| PCD623 | R1480X | Ex 25 | 4438C > T | R1480X | Ex 25 | yes |
| PCD627 | R1480X | Ex 25 | 4438C > T | R1480X | Ex 25 | yes |
| PCD761 | Y759_E889del | Int 13 | 13213delC | R4405AfsX1 | Ex 81 | yes |
| PCD812 | G1939R | Ex 34 | 13373C > T | P4458L | Ex 82 | yes |
| PCD918 | 4356delL | Ex 80 | 13075C > T | R4359X | Ex 80 | yes |
| PCD919 | 4356delL | Ex 80 | 13075C > T | R4359X | Ex 80 | yes |
| PCD974 | L3255S | Ex 60 | no second hit | no second hit | ? | yes |
| PCD998 | K3038TfsX13 | Ex 56 | no second hit | no second hit | ? | yes |
| PCD1022 | R1445X | Ex 24 | 4333C > T | R1445X | Ex 24 | yes |
| PCD1023 | R1445X | Ex 24 | 4333C > T | R1445X | Ex 24 | yes |
| PCD1033 | Q3442X | Ex 63 | no second hit | no second hit | ? | yes |
| PCD1077 | E1301X | Ex 21 | 11804C > T | P3935L | Ex 72 | no |
| PCD1126 | A4022P | Ex 74 | 13504_13505insGAAGA | T4502RfsX14 | Ex 82 | no |
| PCD1174 | R857X | Ex 14 | no second hit | no second hit | ? | yes |
| OP-98 II1 | W2604X | Ex 48 | 13333_13334insACCA | I4445NfsX3 | Ex 82 | yes |
| OP-98 II2 | W2604X | Ex 48 | 13333_13334insACCA | I4445NfsX3 | Ex 82 | yes |
| OP-41 II1 | E117V splice? | Ex 1 | 7148T > C | L2383P | Ex 44 | no |
| OP-235 II1 | Q4233X | Ex 77 | 12980T > C | L4327S | Ex 79 | yes |
| OP-235 II2 | Q4233X | Ex 77 | 12980T > C | L4327S | Ex 79 | yes |
| *OP-20 II1 | R3888H | Ex 71 | 11663G > A | R3888H | Ex 71 | no |
| OP-406 II1 | E1366_G1418del | Int 23 | IVS26 − 1G > A | E1576AfsX4 | Int 26 | yes |
| OP-406 II2 | E1366_G1418del | Int 23 | IVS26 − 1G > A | E1576AfsX4 | Int 26 | yes |

*Consanguineous families
SS = Situs solitus,
SI = Situs inversus,
SA = Situs Ambiguus
F = female,
M = male
na = not available
nNO = nasal nitric oxide measured in nl/min
EM = Electron microscopy
Families with multiple affected are highlighted in grey
DA = dynein arms
Neo RDS = neonatal respiratory distress
sib normal DA = EM was not available for the patient but affected sibling had normal DA.
**When available mutations were analyzed in parents and family members to decipher if both mutations in patient were on the different chromosomes.

TABLE 2
Table 2: List of Mutations in DNAH11 gene

| | | | | | Population Study | |
|---|---|---|---|---|---|---|
| | Amino-Acid | Exon/ | Mutation | | in Disease-free | PCD Patient# |
| Base Change | Residue Change | Intron | Classification | Comments | Subjects | with Mutation |
| 350A > T | E117V | Exon 1 | Possible | 80% conserved splice site (2nd | 0/98 alleles | OP41 II:1 |
| | | | splice | last base of exon) | | |
| IVS13 − 1G > C | Y759_E889del | Intron 13 | Splice | 100% conserved splice site, in | no need | PCD761 |
| (c. 2275 − 1G > C) | | | | vitro RNA analysis show deletion | | |
| | | | | of an exon 14 | | |
| 2569C > T | R857X | Exon 14 | Non-sense | loss of function mutation | no need | PCD1174 |
| 3901G > T | E1301X | Exon 21 | Non-sense | loss of function mutation | no need | PCD1077 |
| IVS23 + 5G > T | E1366_G1418del | Intron 23 | Splice | 84% conserved splice site, in | 0/52 alleles | OP406 II:1 & II:2 |
| (c. 4254 + 5G > T) | | | | vitro RNA analysis show deletion | | |
| | | | | of exon 23 | | |
| 4333C > T | R1445X | Exon 24 | Non-sense | loss of function mutation | no need | PCD1022 & 1023 |
| 4438C > T | R1480X | Exon 25 | Non-sense | loss of function mutation | no need | PCD623 & 627 |
| 4516_4517delCT | L1506SfsX10 | Exon 26 | Frame-shift | loss of function mutation | no need | PCD106 & 108 |
| IVS26 − 1G > A | E1576AfsX4 | Intron 26 | Splice | 100% conserved splice site, in | no need | OP406 II:1 & II:2 |
| (c. 4726 − 1G > A) | | | | vitro RNA analysis show deletion | | |
| | | | | of an exon 27 causing frame- | | |
| | | | | shift | | |
| IVS33 + 1G > A | V1821TfsX7 | Intron 33 | Splice | 100% conserved splice site, in | no need | PCD565 |
| (c. 5778 + 1G > A) | | | | vitro RNA analysis show deletion | | |
| | | | | of exons 32-32 causing frame- | | |
| | | | | shift | | |
| 5815G > A | G1939R | Exon 34 | Missense | non-synonymous, 100% | 0/114 alleles | PCD812 |
| | | | | conserved, intolerant change in | | |
| | | | | 1st AAA module | | |
| 6244C > T | R2082X | Exon 37 | Non-sense | loss of function mutation | no need | PCD157 |
| 7148T > C | L2383P | Exon 44 | Missense | non-synonymous, 100% | 0/116 alleles | OP41 II:1 |
| | | | | conserved, intolerant change | | |
| IVS44 + 1G > A | T2379_Q2422del | Intron 44 | Possible | 100% conserved splice site, in | no need | PCD106 & 108 |
| (c. 7266 + 1G > A) | | | splice | silico program show 100% | | |
| | | | | abrogation of splice site | | |
| 7914G > C | W2604X splice | Exon 48 | Splice | 73% conserved splice site, in | no need | OP98 II:1 & II:2 |
| | | | | vitro RNA analysis show deletion | | |
| | | | | of exon 48 causing stop codon | | |
| 9113_9116delAAGA | K3038TfsX13 | Exon 56 | Frame-shift | loss of function mutation | no need | PCD998 |
| 9764T > C | L3255S | Exon 60 | Missense | non-synonymous, 90% | 0/116 alleles | PCD974 |
| | | | | conserved in microtubule binding | | |
| | | | | site | | |
| 10324C > T | Q3442X | Exon 63 | Non-sense | loss of function mutation | no need | PCD1033 |
| 11663G > A | R3888H | Exon 71 | Missense | non-synonymous, 100% | 0/110 alleles | OP20 II:1 |
| | | | | conserved residue | | |
| 11804C > T | P3935L | Exon 72 | Missense | non-synonymous, 100% | 0/104 alleles | PCD1077 |
| | | | | conserved residue in 6th AAA | | |
| | | | | module | | |
| 11929G > T | E3977X | Exon 73 | Non-sense | loss of function mutation | no need | PCD157 |
| 12064G > C | A4022P | Exon 74 | Missense | non-synonymous, 90% | 0/112 alleles | PCD1126 |
| | | | | conserved residue in 6th AAA | | |
| | | | | module | | |
| 12697C > T | Q4233X | Exon 77 | Non-sense | loss of function mutation | no need | OP235 II:1 & II:2 |
| 12980T > C | L4327S | Exon 79 | Missense | non-synonymous, 100% | 0/118 alleles | OP235 II:1 & II:2 |
| | | | | conserved residue | | |
| 13061T > A | L4354H | Exon 80 | Missense | non-synonymous, 90% | 0/114 alleles | PCD565 |
| | | | | conserved residue | | |
| 13065_13067delCCT | 4356delL | Exon 80 | Frame-shift | loss of function mutation | no need | PCD918 & 919 |
| 13075C > T | R4359X | Exon 80 | Non-sense | loss of function mutation | no need | PCD918 & 919 |
| 13213delC | R4405AfsX1 | Exon 81 | Frame-shift | loss of function mutation | no need | PCD761 |
| 13333_13334insACCA | I4445NfsX3 | Exon 82 | Frame-shift | loss of function mutation | no need | OP98 II:1 & II:2 |
| 13504_13505insGAAGA | T4502RfsX14 | Exon 82 | Frame-shift | loss of function mutation | no need | PCD1126 |
| 13373C > T | P4458L | Exon 82 | Missense | non-synonymous, 100% | 0/108 alleles | PCD812 |
| | | | | conserved residue | | |

TABLE 3
Table 3: List of Mutations in DNAH11 gene based on the Ensembl ENSG00000105877
sequence and the updated sequence described herein

| | | | | Amino-Acid | |
|---|---|---|---|---|---|
| | Amino-Acid | Exon/ | Base change | Residue change | Exon/Intron |
| | Residue Change | Intron | (Ensembl | (Ensembl | (Ensembl |
| Base Change | (updated | (updated | ENSG00000105877 | ENSG00000105877 | ENSG00000105877 |
| (updated sequence) | sequence) | sequence) | sequence) | sequence) | sequence) |
| 350A > T | E117V | Exon 1 | 350A > T | E117V | Exon 1 |
| IVS13 − 1G > C | Y759_E889del | Intron 13 | IVS13 − 1G > C | Y759_E889del | Intron 13 |
| (c. 2275 − 1G > C) | | | (c. 2275 − 1G > C) | | |
| 2569C > T | R857X | Exon 14 | 2569C > T | R857X | Exon 14 |
| 3901G > T | E1301X | Exon 21 | 3901G > T | E1301X | Exon 21 |
| IVS23 + 5G > T | E1366_G1418del | Intron 23 | IVS23 + 5G > T | E1371_G1423del | Intron 23 |
| (c. 4254 + 5G > T) | | | (c. 4269 + 5G > T) | | |
| 4333C > T | R1445X | Exon 24 | 4348C > T | R1450X | Exon 24 |
| 4438C > T | R1480X | Exon 25 | 4453C > T | R1485X | Exon 25 |
| 4516_4517delCT | L1506SfsX10 | Exon 26 | 4531_4532delCT | L1511SfsX10 | Exon 26 |
| IVS26 − 1G > A | E1576AfsX4 | Intron 26 | IVS26 − 1G > A | E1581AfsX4 | Intron 26 |
| (c. 4726 − 1G > A) | | | (c. 4741 − 1G > A) | | |
| IVS33 + 1G > A | V1821TfsX7 | Intron 33 | IVS34 + 1G > A | C1868IfsX20 | Intron 34 |
| (c. 5778 + 1G > A) | | | (c. 5799 + 1G > A) | | |
| 5815G > A | G1939R | Exon 34 | 5836G > A | G1946R | Exon 35 |
| 6244C > T | R2082X | Exon 37 | 6265C > T | R2089X | Exon 38 |
| 7148T > C | L2383P | Exon 44 | 7169T > C | L2390P | Exon 45 |
| IVS44 + 1G > A | T2379_Q2422del | Intron 44 | IVS45 + 1G > A | T2379_Q2422del | Intron 45 |
| (c. 7266 + 1G > A) | | | (c. 7287 + 1G > A) | | |
| 7914G > C | W2604X splice | Exon 48 | 7935G > C | W2604X splice | Exon 49 |
| 9113_9116delAAGA | K3038TfsX13 | Exon 56 | 9134_9137delAAGA | K3045TfsX13 | Exon 57 |
| 9764T > C | L3255S | Exon 60 | 9785T > C | L3262S | Exon 61 |
| 10324C > T | Q3442X | Exon 63 | 10345C > T | Q3449X | Exon 64 |
| 11663G > A | R3888H | Exon 71 | 11684G > A | R3895H | Exon 72 |
| 11804C > T | P3935L | Exon 72 | 11825C > T | P3942L | Exon 73 |
| 11929G > T | E3977X | Exon 73 | 11950G > T | E3984X | Exon 74 |
| 12064G > C | A4022P | Exon 74 | 12085G > C | A4029P | Exon 75 |
| 12697C > T | Q4233X | Exon 77 | 12718C > T | Q4240X | Exon 78 |
| 12980T > C | L4327S | Exon 79 | 13001T > C | L4334S | Exon 80 |
| 13061T > A | L4354H | Exon 80 | 13082T > A | L4361H | Exon 81 |
| 13065_13067delCCT | 4356delL | Exon 80 | 13086_13088delCCT | 4363delL | Exon 81 |
| 13075C > T | R4359X | Exon 80 | 13096C > T | R4366X | Exon 81 |
| 13213delC | R4405AfsX1 | Exon 81 | 13234delC | R4412AfsX1 | Exon 82 |
| 13333_13334insACCA | I4445NfsX3 | Exon 82 | 13354_13355insACCA | I4452NfsX3 | Exon 83 |
| 13504_13505insGAAGA | T4502RfsX14 | Exon 82 | 13525_13526insGAAGA | T4509RfsX14 | Exon 83 |
| 13373C > T | P4458L | Exon 82 | 13394C > T | P4465L | Exon 83 |

TABLE 4
REFERENCE SEQUENCES FOR DNAH11 GENE AND DNAH11 GENE PRODUCT
FOR PCD MUTATIONS 1

| | | |
|---|---|---|
| −31 | CCGGCCTCGCGTTCCCTCGGACGGTTGCCCAATGGCAGCCCAGGTGGCAGCCCGGGAGGC | Exon 1 |
| | ...............................-M--A--A--Q--V--A--A--R--E--A | |
| 30 | GCGAGACTTCAGAGAAGCCCCGACCCTTCGCCTAACCTCGGGGGCCGGCCTGGAGGCAGT | |
| 11 | --R--D--F--R--E--A--P--T--L--R--L--T--S--G--A--G--L--E--A--V | |
| 90 | GGGCGCTGTGGAGCTCGAGGAGGAGGAGGAGAACGAGGAGGAGGCGGCGGCCAGGAGAGC | |
| 31 | --G--A--V--E--L--E--E--E--E--E--N--E--E--E--A--A--A--R--R--A | |
| 150 | GCGGAGTTTCGCCCAAGACGCGCGGGTGCGCTTCCTCGGCGGCCGCCTGGCGATGATGCT | |
| 51 | --R--S--F--A--Q--D--A--R--V--R--F--L--G--G--R--L--A--M--M--L | |
| 210 | GGGGTTCACGGAGGAGAAATGGAGCCAGTATTTGGAAAGCGAGGACAACCGGCAGGTTCT | |
| 71 | --G--F--T--E--E--K--W--S--Q--Y--L--E--S--E--D--N--R--Q--V--L | |
| 270 | TGGGGAGTTTCTGGAAAGCACCAGCCCGGCTTGCCTTGTGTTTAGCTTCGCCGCCTCGGG | |
| 91 | --G--E--F--L--E--S--T--S--P--A--C--L--V--F--S--F--A--A--S--G | |
| | 350A > T[E117V]possible splice mutation | |
| 330 | GCGCCTTGCGGCTTCCCAGGAGATTCCAAGAGATGCAAACCATAAACTTGTTTTTATTTC | Exon 2 |
| 111 | --R--L--A--A--S--Q--E--I--P--R--D--A--N--H--K--L--V--F--I--S | |
| 390 | CAAGAAGATTACTGAAAGCATTGGAGTAAATGACTTTTCTCAAGTGGTTTTATTTGGAGA | |
| 131 | --K--K--I--T--E--S--I--G--V--N--D--F--S--Q--V--V--L--F--G--E | |
| 450 | GTTACCTGCGTTGTCTCTTGGACATGTATCTGCTTTCCTTGATGAGATTTTAGTGCCAGT | Exon 3 |
| 151 | --L--P--A--L--S--L--G--H--V--S--A--F--L--D--E--I--L--V--P--V | |
| 510 | TCTTTCTAATAAGAACAACCATAAGTCCTGGTCCTGTTTTACTTCACAAGATATGGAATA | |
| 171 | --L--S--N--K--N--N--H--K--S--W--S--C--F--T--S--Q--D--M--E--Y | |
| 570 | TCACATAGAAGTCATGAAAAAGAAGATGTATATTTTTAGGGGCAAAATGTCTAGAAGAAC | |
| 191 | --H--I--E--V--M--K--K--K--M--Y--I--F--R--G--K--M--S--R--R--T | |
| 630 | TCTTCTACCAATTCCCACTGTTGCAGGAAAGATGGATCTGGATCAGAATTGTTCAGAGAA | |
| 211 | --L--L--P--I--P--T--V--A--G--K--M--D--L--D--Q--N--C--S--E--N | |
| 690 | CAAGCCACCGTCAAACGAAAGGATAATACTTCATGCAATTGAATCTGTGGTTATTGAATG | Exon 4 |
| 231 | --K--P--P--S--N--E--R--I--I--L--H--A--I--E--S--V--V--I--E--W | |
| 750 | GTCACATCAAATCCAAGAAATTATAGAAAGAGATTCAGTGCAGCGTTTGTTGAATGGTCT | |
| 251 | --S--H--Q--I--Q--E--I--I--E--R--D--S--V--Q--R--L--L--N--G--L | |
| 810 | TCACTTGTCTCCTCAAGCAGAACTAGATTTCTGGATGATGAGGAGAGAAAATCTGTCATG | |
| 271 | --H--L--S--P--Q--A--E--L--D--F--W--M--M--R--R--E--N--L--S--C | |
| 870 | CATTTATGATCAACTTCAGGCACCTGTTGTCCTCAAAATGGTTAAGATCCTGACAACTAA | Exon 5 |
| 291 | --I--Y--D--Q--L--Q--A--P--V--V--L--K--M--V--K--I--L--T--T--K | |
| 930 | ACAAAGCAGCTATTTTCCTACTCTGAAGGACATTTTTCTGGCTGTGGAAAATGCTCTTCT | Exon 6 |
| 311 | --Q--S--S--Y--F--P--T--L--K--D--I--F--L--A--V--E--N--A--L--L | |
| 990 | CGAAGCCCAAGATGTGGAACTTTACCTGAGACCTCTGAGGAGACACATCCAGTGTCTCCA | |
| 331 | --E--A--Q--D--V--E--L--Y--L--R--P--L--R--R--H--I--Q--C--L--Q | |
| 1050 | GGAGACGGAATTCCCACAGACACGCATATTAATCGCTCCATTATTTCATACCATCTGTCT | |
| 351 | --E--T--E--F--P--Q--T--R--I--L--I--A--P--L--F--H--T--I--C--L | |
| 1110 | GATCTGGAGTCATTCCAAGTTTTATAACACCCCAGCTCGGGTTATAGTTTTATTGCAAGA | |
| 371 | --I--W--S--H--S--K--F--Y--N--T--P--A--R--V--I--V--L--L--Q--E | |
| 1170 | GTTTTGTAATCTCTTCATTAACCAGGCAACAGCTTACCTTTCACCTGAGGACCTTTTGAG | Exon 7 |
| 391 | --F--C--N--L--F--I--N--Q--A--T--A--Y--L--S--P--E--D--L--L--R | |
| 1230 | GGGAGAAATAGAAGAGTCACTGGAAAAGGTGCAGGTGGCTGTTAACATCTTAAAGACTTT | |
| 411 | --G--E--I--E--E--S--L--E--K--V--Q--V--A--V--N--I--L--K--T--F | |
| 1290 | CAAAAACTCCTTTTTCAACTATAGAAAAAAATTGGCAAGCTACTTTATGGGAAGAAAGCT | |
| 431 | --K--N--S--F--F--N--Y--R--K--K--L--A--S--Y--F--M--G--R--K--L | |
| 1350 | GAGACCATGGGATTTCCAGTCTCATCTGGTGTTTTGCAGATTTGACAAGTTTCTTGATCG | |
| 451 | --R--P--W--D--F--Q--S--H--L--V--F--C--R--F--D--K--F--L--D--R | |
| 1410 | TTTAATAAAAATAGAGGATATATTTGCCACCACTTTGGAATTTGAAAAGCTGGAAAGACT | Exon 8 |
| 471 | --L--I--K--I--E--D--I--F--A--T--T--L--E--F--E--K--L--E--R--L | |
| 1470 | GGAATTTGGTGGTACCAAAGGAGCAATTTTAAATGGACAAGTCCACGAGATGAGTGAAGA | |
| 491 | --E--F--G--G--T--K--G--A--I--L--N--G--Q--V--H--E--M--S--E--E | |
| 1530 | ACTTATGGAACTCTGTAAACTTTTTAAACAGAGCACTTATGACCCATCTGATTGCACTAA | |
| 511 | --L--M--E--L--C--K--L--F--K--Q--S--T--Y--D--P--S--D--C--T--N | |
| 1590 | CATGGAGTTTGAAAGTGATTATGTGGCATTTAAGTCCAAAACTCTGGAATTTGACAGAAG | Exon 9 |
| 531 | --M--E--F--E--S--D--Y--V--A--F--K--S--K--T--L--E--F--D--R--R | |
| 1650 | GCTTGGGACAATTATTTGTGAAGCTTTCTTTAACTGCAATGGCTTAGAAGCTGCATTTAA | |
| 551 | --L--G--T--I--I--C--E--A--F--F--N--C--N--G--L--E--A--A--F--K | |
| 1710 | GCTTTTGACCATATTTGGAAATTTTCTAGAGAAGCCAGTTGTCATGGAAATTTTCAGCCT | Exon 10 |
| 571 | --L--L--T--I--F--G--N--F--L--E--K--P--V--V--M--E--I--F--S--L | |
| 1770 | ACATTACAGCACACTAGTGCATATGTTTAATACAGAGCTGGATGTGTGTAAGCAACTGTA | |
| 591 | --H--Y--S--T--L--V--H--M--F--N--T--E--L--D--V--C--K--Q--L--Y | |
| 1830 | TAATGAACACATGAAACAGATTGAATGTGGTCATGTAGTTCTTAACAAGAACATGCCATT | Exon 11 |
| 611 | --N--E--H--M--K--Q--I--E--C--G--H--V--V--L--N--K--N--M--P--F | |
| 1890 | TACCTCAGGAAATATGAAATGGGCCCAGCAGGTTCTCCAACGACTTCAAATGTTTTGGTC | |
| 631 | --T--S--G--N--M--K--W--A--Q--Q--V--L--Q--R--L--Q--M--F--W--S | |
| 1950 | AAACTTCGCATCTCTCCGTTATCTATTTTTGGGCAATCCTGATCACGCTTTAGTTTATCA | Exon 12 |
| 651 | --N--F--A--S--L--R--Y--L--F--L--G--N--P--D--H--A--L--V--Y--Q | |
| 2010 | AAAGTATGTTGAAATGACCACTTTGCTTGATCAATTTGAAAGTCGTATCTATAATGAATG | |
| 671 | --K--Y--V--E--M--T--T--L--L--D--Q--F--E--S--R--I--Y--N--E--W | |
| 2070 | GAAAAGTAATGTGGATGAAATCTGTGAATTCAATTTGAATCAACCCTTGGTTAAATTCAG | |
| 691 | --K--S--N--V--D--E--I--C--E--F--N--L--N--Q--P--L--V--K--F--S | |
| 2130 | TGCCATAAATGGTCTTCTCTGTGTCAATTTTGACCCAAAGCTAGTGGCTGTATTGAGAGA | Exon 13 |
| 711 | --A--I--N--G--L--L--C--V--N--F--D--P--K--L--V--A--V--L--R--E | |
| 2190 | AGTGAAATATCTTTTGATGTTGAAGAAACAAGACATACCAGATTCAGCTTTAGCCATCTT | |
| 731 | --V--K--Y--L--L--M--L--K--K--Q--D--I--P--D--S--A--L--A--I--F | |
| 2250 | CAAGAAAAGGAACACTATTTTAAAGTACATTGGAAATCTTGACCTTCTTGTGCAAGGGTA | Exon 14 |
| 751 | --K--K--R--N--T--I--L--K--Y--I--G--N--L--D--L--L--V--Q--G--Y | |
| 2310 | TAATAAACTCAAACAGACGCTCCTGGAAGTTGAATACCCTCTGATTGAAGATGAGCTGAG | |
| 771 | --N--K--L--K--Q--T--L--L--E--V--E--Y--P--L--I--E--D--E--L--R | |
| 2370 | GGCTATTGACGAGCAGCTGACAGCAGCCACAACGTGGCTGACATGGCAGGATGACTGCTG | |
| 791 | --A--I--D--E--Q--L--T--A--A--T--T--W--L--T--W--Q--D--D--C--W | |
| 2430 | GGGCTACATCGAGAGGGTGAGGGCAGCCACGTCCGAGTTGGAGCACAGAGTTGAGCGCAC | |
| 811 | --G--Y--I--E--R--V--R--A--A--T--S--E--L--E--H--R--V--E--R--T | |
| 2490 | ACAGAAAAACGTGAAGGTGATCCAGCAGACCATGAGGGGCTGGGCCAGGTGCGTGCTACC | |
| 831 | --Q--K--N--V--K--V--I--Q--Q--T--M--R--G--W--A--R--C--V--L--P | |
| | 2569C > T[R857X] | |
| 2550 | TCCCAGGAGAGAGCACAGACGAGAGGCAGCCTTCACCTTGGAGGACAAGGGTGATTTGTT | |
| 851 | --P--R--R--E--H--R--R--E--A--A--F--T--L--E--D--K--G--D--L--F | |
| 2610 | TACAAAAAAATACAAGTTAATCCAAGGAGATGGCTGCAAGATCCACAACTTGGTCGAGGA | Exon 15 |
| 871 | --T--K--K--Y--K--L--I--Q--G--D--G--C--K--I--H--N--L--V--E--E | |
| 2670 | AAATAGGAAGCTCTTCAAAGCCAATCCCTCTCTGGATACCTGGAAAATTTATGTAGAATT | |
| 891 | --N--R--K--L--F--K--A--N--P--S--L--D--T--W--K--I--Y--V--E--F | |
| 2730 | CATTGACGACATTGTGGTGGAAGGCTTTTTTCAGGCTATAATGCACGACTTAGACTTCTT | |
| 911 | --I--D--D--I--V--V--E--G--F--F--Q--A--I--M--H--D--L--D--F--F | |
| 2790 | TCTGAAGAATACAGAGAAACAATTGAAACCGGCACCGTTTTTTCAAGCACAAATGATCTT | |
| 931 | --L--K--N--T--E--K--Q--L--K--P--A--P--F--F--Q--A--Q--M--I--L | |
| 2850 | GTTGCCTCCTGAGATTGTGTTTAAACCTTCCCTAGACAGAGAGGCTGGGGATGGCTTCTA | |
| 951 | --L--P--P--E--I--V--F--K--P--S--L--D--R--E--A--G--D--G--F--Y | |
| 2910 | TGATCTTGTAGAAGAAATGTTATGCAATAGTTTTAGAATGTCTGCCCAGATGAACCGAAT | |
| 971 | --D--L--V--E--E--M--L--C--N--S--F--R--M--S--A--Q--M--N--R--I | |
| 2970 | AGCAACACACCTGGAAATTAAAAATTATCAGAATGATATGGATAACATGTTAGGCCTGGC | Exon 16 |
| 991 | --A--T--H--L--E--I--K--N--Y--Q--N--D--M--D--N--M--L--G--L--A | |
| 3030 | AGAGGTCAGGCAGGAGATCATGAACAGAGTGGTGAATGTCATCAACAAAGTCTTAGATTT | |
| 1011 | --E--V--R--Q--E--I--M--N--R--V--V--N--V--I--N--K--V--L--D--F | |
| 3090 | CAGAAACACCCTGGAGACCCACACTTACCTCTGGGTGGATGATCGAGCTGAGTTTATGAA | |
| 1031 | --R--N--T--L--E--T--H--T--Y--L--W--V--D--D--R--A--E--F--M--K | |
| 3150 | GCATTTTCTCTTGTATGGCCATGCTGTGTCTTCCGATGAAATGGATGCTCATGCAAATGA | |
| 1051 | --H--F--L--L--Y--G--H--A--V--S--S--D--E--M--D--A--H--A--N--E | |
| 3210 | AGAAATTCCCGAACAACCACCAACTCTTGAGCAATTCAAAGAACAGATTGACATTTATGA | Exon 17 |
| 1071 | --E--I--P--E--Q--P--P--T--L--E--Q--F--K--E--Q--I--D--I--Y--E | |
| 3270 | AGCTTTGTATGTTCAAATGAGCAAATTTGAGGACTTTAGAGTGTTTGATAGTTGGTTCAA | |
| 1091 | --A--L--Y--V--Q--M--S--K--F--E--D--F--R--V--F--D--S--W--F--K | |
| 3330 | GGTGGACATGAAGCCTTTCAAAGTGAGCTTGTTAACCATAATTAAGAAATGGAGCTGGAT | |
| 1111 | --V--D--M--K--P--F--K--V--S--L--L--T--I--I--K--K--W--S--W--M | |
| 3390 | GTTTCAGGAGCATCTTTTGAGATTTGTCATTGACAGTCTGAATGAGCTACAAGAATTTAT | Exon 18 |
| 1131 | --F--Q--E--H--L--L--R--F--V--I--D--S--L--N--E--L--Q--E--F--I | |
| 3450 | AAAGGAGACAGATTCCGGACTTCAGAGAGAATTAAATGAAGGTGATCATGATGGTTTAGT | |
| 1151 | --K--E--T--D--S--G--L--Q--R--E--L--N--E--G--D--H--D--G--L--V | |
| 3510 | TGACATCATGGTGCATCTTCTGGCTGTAAGAAGCCGACAGAGAGCTACTGATGAACTCTT | |
| 1171 | --D--I--M--V--H--L--L--A--V--R--S--R--Q--R--A--T--D--E--L--F | |
| 3570 | TGAACCTCTAAAAGAAACGATCACCCTCTTGGAAAGCTATGGCCAGAAGATGCCTGAGCA | |
| 1191 | --E--P--L--K--E--T--I--T--L--L--E--S--Y--G--Q--K--M--P--E--Q | |
| 3630 | GGTCTATATTCAGCTAGAGGAATTACCTGAAAGATGGGAAACTACCAAAAAGATCGCAGC | Exon 19 |
| 1211 | --V--Y--I--Q--L--E--E--L--P--E--R--W--E--T--T--K--K--I--A--A | |
| 3690 | AACTGTCAGACATGAAGTCTCACCTCTCCATAATGCGGAAGTCACTCTTATAAGGAAAAA | |
| 1231 | --T--V--R--H--E--V--S--P--L--H--N--A--E--V--T--L--I--R--K--K | |
| 3750 | ATGTATTTTGTTTGACGCAAAGCAGGCAGAGTTCAGAGAGAGATTCAGACACTATGCCCC | Exon 20 |
| 1251 | --C--I--L--F--D--A--K--Q--A--E--F--R--E--R--F--R--H--Y--A--P | |
| 3810 | TCTTGGATTTAATGCAGAAAATCCATACACAGCGCTTGATAAGGCAAATGAAGAGCTTGA | Exon 21 |
| 1271 | --L--G--F--N--A--E--N--P--Y--T--A--L--D--K--A--N--E--E--L--E | |
| | 3901G > T[E1301X] | |
| 3870 | GGCCTTAGAAGAAGAAATGTTGCAGATGCAAGAATCTACTCGTCTTTTTGAAGTGGCTCT | |
| 1291 | --A--L--E--E--E--M--L--Q--M--Q--E--S--T--R--L--F--E--V--A--L | |
| 3930 | TCCAGAGTACAAACAAATGAAACAGTGTCGCAAAGAAATAAAATTGCTCAAGGGACTGTG | |
| 1311 | --P--E--Y--K--Q--M--K--Q--C--R--K--E--I--K--L--L--K--G--L--W | |
| 3990 | GGATGTCATTATTTATGTTCGAAGAAGCATTGATAATTGGACTAAAACCCAGTGGAGACA | Exon 22 |
| 1331 | --D--V--I--I--Y--V--R--R--S--I--D--N--W--T--K--T--Q--W--R--Q | |
| 4050 | GATTCATGTGGAACAGATGGATGTAGAACTCAGAAGGTTTGCCAAG-------------- | |
| 1351 | --I--H--V--E--Q--M--D--V--E--L--R--R--F--A--K--------------- | |
| 4096 | -GAAATTTGGTCACTCAACAAGGAAGTCCGCGTCTGGGATGCTTACACGGGCCTGGAAGG | Exon 23 |
| 1366 | --E--I--W--S--L--N--K--E--V--R--V--W--D--A--Y--T--G--L--E--G | |
| 4155 | CACAGTTAAGGACATGACAGCCTCCCTGAGGGCCATCACAGAGTTACAGAGCCCTGCCCT | |
| 1386 | --T--V--K--D--M--T--A--S--L--R--A--I--T--E--L--Q--S--P--A--L | |
| 4215 | CAGGGACAGGCATTGGCACCAGCTGATGAAAGCTATTG0GGTCAAGTTTTTAATAAATGA | Exon 24 |
| 1406 | --R--D--R--H--W--H--Q--L--M--K--A--I--G--V--K--F--L--I--N--E | |
| | 4333C > T[R1445X] | |
| 4275 | AGCCACAACTTTGGCAGATTTGTTAGCACTGCGGTTACACAGAGTGGAAGATGATGTCCG | |
| 1426 | --A--T--T--L--A--D--L--L--A--L--R--L--H--R--V--E--D--D--V--R | |
| 4335 | AAGGATTGTGGACAAGGCGGTGAAAGAGCTGGGGACTGAGAAGGTTATTACTGAAATCAG | Exon 25 |
| 1446 | --R--I--V--D--K--A--V--K--E--L--G--T--E--K--V--I--T--E--I--S | |
| | 4438C > T[R1480X] | |
| 4395 | TCAGACCTGGGCAACCATGAAGTTTTCTTACGAAGTTCACTATCGAACAGGCATTCCATT | |
| 1466 | --Q--T--W--A--T--M--K--F--S--Y--E--V--H--Y--R--T--G--I--P--L | |
| 4455 | ACTAAAGTCTGATGAACAACTTTTTGAAACTCTAGAGCACAACCAAGTTCAGTTGCAGAC | Exon 26 |
| 1486 | --L--K--S--D--E--Q--L--F--E--T--L--E--H--N--Q--V--Q--L--Q--T | |
| | 4516_4517de1CT[L1506SfsX10] | |
| 4515 | TCTTCTTCAAAGCAAGTATGTAGAATATTTCATTGAGCAAGTGTTAAGCTGGCAAAATAA | |
| 1506 | --L--L--Q--S--K--Y--V--E--Y--F--I--E--Q--V--L--S--W--Q--N--K | |
| 4575 | ATTAAACATAGCAGACTTGGTCATCTTCACTTGGATGGAAGTCCAGCGAACTTGGTCTCA | |
| 1526 | --L--N--I--A--D--L--V--I--F--T--W--M--E--V--Q--R--T--W--S--H | |
| 4635 | CCTGGAAAGCATTTTTGTCTGTTCAGAAGATATTCGAATCCAGCTTGTGAAAGATGCTAG | |
| 1546 | --L--E--S--I--F--V--C--S--E--D--I--R--I--Q--L--V--K--D--A--R | |
| 4695 | AAGATTTGATGGGGTGGATGCTGAATTTAAGGAGTTAATGTTCAAGACAGCCAAAGTAGA | Exon 27 |
| 1566 | --R--F--D--G--V--D--A--E--F--K--E--L--M--F--K--T--A--K--V--E | |
| 4755 | AAATGTGTTAGAAGCAACGTGCAGACCTAATCTCTATGAAAAACTTAAAGATTTACAGTC | |
| 1586 | --N--V--L--E--A--T--C--R--P--N--L--Y--E--K--L--K--D--L--Q--S | |
| 4815 | CAGGCTTTCTCTTTGTGAAAAAGCTCTCGCTGAATACCTGGAAACCAAGCGCATAGCCTT | Exon 28 |
| 1606 | --R--L--S--L--C--E--K--A--L--A--E--Y--L--E--T--K--R--I--A--F | |
| 4875 | TCCTCGCTTCTATTTCGTCTCTTCTGCTGATTTACTTGACATTCTCTCAAAAGGAGCTCA | |
| 1626 | --P--R--F--Y--F--V--S--S--A--D--L--L--D--I--L--S--K--G--A--Q | |
| 4935 | GCCTAAACAGGTAACATGTCACCTTGCCAAACTTTTCGACAGCATTGCAGATCTGCAGTT | Exon 29 |
| 1646 | --P--K--Q--V--T--C--H--L--A--K--L--F--D--S--I--A--D--L--Q--F | |
| 4995 | TGAAGACAATCAGGATGTTTCTGCACACAGGGCAGTTGGAATGTACAGCAAAGAAAAGGA | |
| 1666 | --E--D--N--Q--D--V--S--A--H--R--A--V--G--M--Y--S--K--E--K--E | |
| 5055 | GTATGTCCCATTCCAAGCCGAGTGTGAATGTGTGGGCCATGTGGAAACATGGCTTCTGCA | Exon 30 |
| 1686 | --Y--V--P--F--Q--A--E--C--E--C--V--G--H--V--E--T--W--L--L--Q | |
| 5115 | ACTTGAACAGACTATGCAAGAAACGGTGCGTCATTCTATAACAGAAGCCATAGTGGCCTA | |
| 1706 | --L--E--Q--T--M--Q--E--T--V--R--H--S--I--T--E--A--I--V--A--Y | |
| 5175 | CGAGGAAAAACCTAGGGAACTGTGGATTTTTGATTTCCCAGCTCAGGTTGCACTAACCAG | |
| 1726 | --E--E--K--P--R--E--L--W--I--F--D--F--P--A--Q--V--A--L--T--S | |
| 5235 | CTCACAAATATGGTGGACCACAGATGTAGGAATAGCCTTCAGTAGACTGGAAGAAGGCTA | |
| 1746 | --S--Q--I--W--W--T--T--D--V--G--I--A--F--S--R--L--E--E--G--Y | |
| 5295 | CGAAACAGCCCTGAAGGATTTCCATAAAAAACAGATTTCTCAGCTGAATACACTGATTAC | Exon 31 |
| 1766 | --E--T--A--L--K--D--F--H--K--K--Q--I--S--Q--L--N--T--L--I--T | |
| 5355 | ACTTTTGCTGGGAGAACTTCCACCTGGAGACAGACAGAAGATCATGACAATTTGTACCAT | |
| 1786 | --L--L--L--G--E--L--P--P--G--D--R--Q--K--I--M--T--I--C--T--I | |
| 5415 | AGATGTCCATGCCAGAGACGTGGTGGCAAAACTTATTTCTCAGAAG------GTTGTCAG | Exon 32 |
| 1806 | --D--V--H--A--R--D--V--V--A--K--L--I--S--Q--K--------V--V--S | |
| 5469 | TCCCCAAGCTTTTACATGGCTGTCTCAACTTCGTCACCGATGGGAGGATACCCAGAAACA | |
| 1824 | --P--Q--A--F--T--W--L--S--Q--L--R--H--R--W--E--D--T--Q--K--H | |
| 5529 | CTGCTTTGTTAATATTTGTGATGCCCAGTTCCAGTACTTCTATGAATACTTAGGAAACAG | |
| 1844 | --C--F--V--N--I--C--D--A--Q--F--Q--Y--F--Y--E--Y--L--G--N--S | |
| 5589 | CCCTCGACTAGTGATCACTCCTCTAACTGACAGGTGTTATATTACCTTAACTCAATCACT | Exon 33 |
| 1864 | --P--R--L--V--I--T--P--L--T--D--R--C--Y--I--T--L--T--Q--S--L | |
| 5649 | TCATCTAACCATGAGTGGGGCTCCTGCTGGCCCAGCTGGTACCGGGAAAACAGAGACCAC | |
| 1884 | --H--L--T--M--S--G--A--P--A--G--P--A--G--T--G--K--T--E--T--T | |
| 5709 | CAAAGACCTAGGACGTGCCCTTGGCATGATGGTCTATGTATTCAACTGTTCAGAGCAAAT | |
| 1904 | --K--D--L--G--R--A--L--G--M--M--V--Y--V--F--N--C--S--E--Q--M | |
| | 5815G > A[G1939R] | |
| 5769 | GGACTACAAATCCATAGGCAATATCTATAAGGGATTGGTGCAGACAGGAGCTTGGGGCTG | Exon 34 |
| 1924 | --D--Y--K--S--I--G--N--I--Y--K--G--L--V--Q--T--G--A--W--G--C | |
| 5829 | CTTTGATGAGTTCAACCGAATCTCTGTGGAAGTTCTGTCAGTGGTGGCAGTACAAGTGAA | |
| 1944 | --F--D--E--F--N--R--I--S--V--E--V--L--S--V--V--A--V--Q--V--K | |
| 5889 | AATGATTCATGATGCCATCAGAAACAGGAAGAAGAGATTTGTATTTCTTGGGGAAGCTAT | Exon 35 |
| 1964 | --M--I--H--D--A--I--R--N--R--K--K--R--F--V--F--L--G--E--A--I | |
| 5949 | CACACTGAAGCCATCAGTTGGAATATTTATTACTATGAACCCGGGTTATGCTGGTCGAAC | |
| 1984 | --T--L--K--P--S--V--G--I--F--I--T--M--N--P--G--Y--A--G--R--T | |
| 6009 | CGAATTACCGGAAAATCTCAAAGCTCTTTTCAGACCCTGTGCCATGGTGGCCCCTGACAT | Exon 36 |
| 2004 | --E--L--P--E--N--L--K--A--L--F--R--P--C--A--M--V--A--P--D--I | |
| 6069 | TGAGCTAATCTGTGAAATCTTGTTAGTTGCTGAAGGTTTTGTGGATGCGCGTGCATTAGC | |
| 2024 | --E--L--I--C--E--I--L--L--V--A--E--G--F--V--D--A--R--A--L--A | |
| 6129 | CCGAAAGTTCATTACGTTGTACACGCTTTGCAAGGAGCTTCTCTCCAAGCAGGATCATTA | Exon 37 |
| 2044 | --R--K--F--I--T--L--Y--T--L--C--K--E--L--L--S--K--Q--D--H--Y | |
| | 6244C > T[R2082X] | |
| 6189 | CGACTGGGGACTTCGTGCTATTAAGTCTGTCTTGGTTGTGGCTGGATCTCTGAAACGAGG | |
| 2064 | --D--W--G--L--R--A--I--K--S--V--L--V--V--A--G--S--L--K--R--G | |
| 6249 | AGATAAAAATAGACCCGAAGATCAGGTACTCATGAGAGCATTAAGGGATTTCAATATGCC | Exon 38 |
| 2084 | --D--K--N--R--P--E--D--Q--V--L--M--R--A--L--R--D--F--N--M--P | |
| 6309 | CAAAATAGTGACTGACGACATCCCAGTGTTTCTGGGCCTGGTCGGTGACCTGTTTCCAGC | |
| 2104 | --K--I--V--T--D--D--I--P--V--F--L--G--L--V--G--D--L--F--P--A | |
| 6369 | CCTGGATGTGCCCCGGAGGAGGAAGCTGCACTTTGAACAGATGGTCAGGCAGTCTACCCT | |
| 2124 | --L--D--V--P--R--R--R--K--L--H--F--E--Q--M--V--R--Q--S--T--L | |
| 6429 | GGAGCTCCGCCTGCAGCCTGAAGAGAGCTTCATCCTCAAAGTTGTCCAGCTTGAGGAACT | Exon 39 |
| 2144 | --E--L--R--L--Q--P--E--E--S--F--I--L--K--V--V--Q--L--E--E--L | |
| 6489 | GTTGGCTGTGCGGCACTCGGTCTTTGTAGTTGGAAATGCAGGCACAGGAAAGAGTAAGAT | Exon 40 |
| 2164 | --L--A--V--R--H--S--V--F--V--V--G--N--A--G--T--G--K--S--K--I | |
| 6549 | TTTGAGAACACTGAACCGAACATATGTTAACATGAAACAGAAGCCGGTTTGGAATGACTT | |
| 2184 | --L--R--T--L--N--R--T--Y--V--N--M--K--Q--K--P--V--W--N--D--L | |
| 6609 | AAACCCTAAAGCTGTGACAACAGATGAACTCTTTGGTTTCATACATCATGCTACCCGAGA | |
| 2204 | --N--P--K--A--V--T--T--D--E--L--F--G--F--I--H--H--A--T--R--E | |
| 6669 | ATGGAAAGATGGCAAGATTGTTTACTCTTATTTTATAGGTCTCTTCTCATCCATTCTACG | Exon 41 |
| 2224 | --W--K--D--G--K--I--V--Y--S--Y--F--I--G--L--F--S--S--I--L--R | |
| 6729 | AGAACAAGCAAATCTTAAGCATGATGGACCAAAATGGATAGTCCTGGATGGCGATATTGA | |
| 2244 | --E--Q--A--N--L--K--H--D--G--P--K--W--I--V--L--D--G--D--I--D | |
| 6789 | CCCCATGTGGATTGAATCACTGAATACTGTAATGGATGATAACAAGGTGCTGACCCTCGC | Exon 42 |
| 2264 | --P--M--W--I--E--S--L--N--T--V--M--D--D--N--K--V--L--T--L--A | |
| 6849 | CAGCAATGAGCGCATTGCACTCACTCCCTTCATGAGGCTTCTGTTTGAGATACATCACTT | |
| 2284 | --S--N--E--R--I--A--L--T--P--F--M--R--L--L--F--E--I--H--H--L | |
| 6909 | AAGGAGCGCAACCCCGGCCACTGTTTCCAGAGCTGGTATTCTGTATGTGAACCCACAAGA | |
| 2304 | --R--S--A--T--P--A--T--V--S--R--A--G--I--L--Y--V--N--P--Q--D | |
| 6969 | TCTGGGCTGGAATCCGTATGTGGCCAGTTGGATAGACAGAAGGCGGCATCAATCAGAAAA | Exon 43 |
| 2324 | --L--G--W--N--P--Y--V--A--S--W--I--D--R--R--R--H--Q--S--E--K | |
| 7029 | GGCCAATTTGACTATTCTTTTTGATAAATATGTCCCTGCATGCTTGGATAAACTGAGAAC | |
| 2344 | --A--N--L--T--I--L--F--D--K--Y--V--P--A--C--L--D--K--L--R--T | |
| | 7148T > C[L2383P] | |
| 7089 | AAGCTTTAAAACCATCACTTCAATTCCTGAGAGTAGCCTGGTGCAGACTCTATGTGTTCT | Exon 44 |
| 2364 | --S--F--K--T--I--T--S--I--P--E--S--S--L--V--Q--T--L--C--V--L | |
| 7149 | TTTGGAGTGCTTGCTGACTCCTGAAAATGTACCTTCTGACAGCCCAAAAGAAGTTTATGA | |
| 2384 | --L--E--C--L--L--T--P--E--N--V--P--S--D--S--P--K--E--V--Y--E | |
| 7209 | AGTCTATTTTGTATTTGCTTGTATCTGGGCTTTTGGAGGCACCCTGCTACAAGATCAGAT | Exon 45 |
| 2404 | --V--Y--F--V--F--A--C--I--W--A--F--G--G--T--L--L--Q--D--Q--I | |
| 7269 | TTCTGATTATCAAGCTGACTTCAGTCGGTGGTGGCAGAAAGAGATGAAAGCAGTGAAATT | |
| 2424 | --S--D--Y--Q--A--D--F--S--R--W--W--Q--K--E--M--K--A--V--K--F | |
| 7329 | TCCGTCGCAGGGAACAATCTTTGATTATTATGTGGACCACAAAACTAAGAAATTATTGCC | |
| 2444 | --P--S--Q--G--T--I--F--D--Y--Y--V--D--H--K--T--K--K--L--L--P | |
| 7389 | CTGGGCTGACAAAATTGCCCAGTTTACTATGGATCCAGATGTGCCTCTGCAGACAGTTCT | Exon 46 |
| 2464 | --W--A--D--K--I--A--Q--F--T--M--D--P--D--V--P--L--Q--T--V--L | |
| 7449 | CGTTCACACAACAGAGACAGCTCGTCTTAGATATTTCATGGAGTTGTTGCTTGAGAAAGG | |
| 2484 | --V--H--T--T--E--T--A--R--L--R--Y--F--M--E--L--L--L--E--K--G | |
| 7509 | AAAACCTCTAATGCTAGTAGGAAATGCAGGAGTGGGAAAAACAGTCTTTGTAGGTGACAC | |
| 2504 | --K--P--L--M--L--V--G--N--A--G--V--G--K--T--V--F--V--G--D--T | |
| 7569 | ATTGGCAAGTCTCTCTGAGGATTACATAGTATCCCGTGTGCCTTTCAACTACTACACGAC | |
| 2524 | --L--A--S--L--S--E--D--Y--I--V--S--R--V--P--F--N--Y--Y--T--T | |
| 7629 | ATCCACAGCTCTGCAAAAAATTCTTGAGAAACCCCTAGAGAAAAAAGCTGGTCATAACTA | Exon 47 |
| 2544 | --S--T--A--L--Q--K--I--L--E--K--P--L--E--K--K--A--G--H--N--Y | |
| 7689 | TGGTCCTGGAGGAAATAAAAAATTGATTTATTTTATCGACGACATGAACATGCCTGAAGT | |
| 2564 | --G--P--G--G--N--K--K--L--I--Y--F--I--D--D--M--N--M--P--E--V | |
| 7749 | GGACTTATATGGCACCGTTCAGCCTCACACCCTGATCCGGCAGCATATTGATTATGGACA | |
| 2584 | --D--L--Y--G--T--V--Q--P--H--T--L--I--R--Q--H--I--D--Y--G--H | |
| 7809 | TTGGTATGATAGACAGAAGGTGATGCTTAAAGAAATCCATAACTGCCAGTATGTCGCCTG | Exon 48 |
| 2604 | --W--Y--D--R--Q--K--V--M--L--K--E--I--H--N--C--Q--Y--V--A--C | |
| | 7914G > C [W2604X] | |
| 7869 | CATGAATCCGATGGTGGGCAGCTTCACCATCAATCCCAGGCTACAGAGACATTTCACAGT | Exon 49 |
| 2624 | --M--N--P--M--V--G--S--F--T--I--N--P--R--L--Q--R--H--F--T--V | |
| 7929 | GTTTGCATTCAATTTTCCATCTTTGGATGCACTAAACACCATCTATGGCCAAATCTTTAG | |
| 2644 | --F--A--F--N--F--P--S--L--D--A--L--N--T--I--Y--G--Q--I--F--S | |
| 7989 | CTTCCATTTCCAACAGCAAGCATTTGCTCCATCAATTCTCAGGAGTGGCCCCACTTTGAT | |
| 2664 | --F--H--F--Q--Q--Q--A--F--A--P--S--I--L--R--S--G--P--T--L--I | |
| 8049 | CCAGGCAACAATAGCATTCCATCAGACAATGATGTGTAACTTTTTACCCACGGCTATTAA | |
| 2684 | --Q--A--T--I--A--F--H--Q--T--M--M--C--N--F--L--P--T--A--I--K | |
| 8109 | ATTCCACTACATCTTTAATCTGAGAGATTTATCAAACGTCTTCCAGGGGATTTTATTTGC | Exon 50 |
| 2704 | --F--H--Y--I--F--N--L--R--D--L--S--N--V--F--Q--G--I--L--F--A | |
| 8169 | TTCTCCTGAGTGTTTAAAAGGTCCACTTGATTTAATACATCTGTGGCTTCATGAATCTGC | |
| 2724 | --S--P--E--C--L--K--G--P--L--D--L--I--H--L--W--L--H--E--S--A | |
| 8229 | CCGTGTTTATGGAGACAAACTGATAGACAAAAAAGATTGTGATTTGTTTCAGAGAAGAAT | |
| 2744 | --R--V--Y--G--D--K--L--I--D--K--K--D--C--D--L--F--Q--R--R--M | |
| 8289 | GCTGGAAACTGCTTATAAATATTTTGAAGGTATAGATAGTCACATGCTGCTTCAACAGCC | Exon 51 |
| 2764 | --L--E--T--A--Y--K--Y--F--E--G--I--D--S--H--M--L--L--Q--Q--P | |
| 8349 | CCTCATTTATTGCCACTTTGCTGATAGAGGGAAGGACCCACATTACATGCCAGTGAAGGA | |
| 2784 | --L--I--Y--C--H--F--A--D--R--G--K--D--P--H--Y--M--P--V--K--D | |
| 8409 | CTGGGAAGTGCTGAAGACGATTCTTACAGAAACGTTAGACAACTACAATGAACTAAATGC | |
| 2804 | --W--E--V--L--K--T--I--L--T--E--T--L--D--N--Y--N--E--L--N--A | |
| 8469 | TGCCATGCACCTAGTTTTGTTTGAAGATGCCATGCAACATGTGTGTCGCATCAGCCGGAT | Exon 52 |
| 2824 | --A--M--H--L--V--L--F--E--D--A--M--Q--H--V--C--R--I--S--R--I | |
| 8529 | CTTACGAACCCCTCAGGGCTGTGCTCTCTTGGTTGGAGTTGGGGGCAGTGGCAAGCAGAG | |
| 2844 | --L--R--T--P--Q--G--C--A--L--L--V--G--V--G--G--S--G--K--Q--S | |
| 8589 | CTTGTCCAGGCTGGCAGCTTACCTTCGTGGCCTTGAGGTCTTTCAGATCACTCTGACCGA | |
| 2864 | --L--S--R--L--A--A--Y--L--R--G--L--E--V--F--Q--I--T--L--T--E | |
| 8649 | GGGCTATGGAATCCAGGAACTTCGGGTAGATCTTGCCAATTTGTACATCCGAACTGGAGC | Exon 53 |
| 2884 | --G--Y--G--I--Q--E--L--R--V--D--L--A--N--L--Y--I--R--T--G--A | |
| 8709 | CAAGAACATGCCCACTGTGTTCCTGCTGACAGATGCCCAGGTTCTAGATGAGAGCTTCCT | |
| 2904 | --K--N--M--P--T--V--F--L--L--T--D--A--Q--V--L--D--E--S--F--L | |
| 8769 | CGTGCTGATTAATGACTTGCTGGCATCAGGAGAAATCCCAGATCTGTTCAGCGATGAAGA | Exon 54 |
| 2924 | --V--L--I--N--D--L--L--A--S--G--E--I--P--D--L--F--S--D--E--D | |
| 8829 | TGTGGACAAGATAATTTCTGGAATTCATAATGAAGTTCATGCTCTGGGCATGGTAGACTC | |
| 2944 | --V--D--K--I--I--S--G--I--H--N--E--V--H--A--L--G--M--V--D--S | |
| 8889 | CAGGGAAAACTGTTGGAAATTCTTTATGGCCAGGGTGCGACTACAGCTCAAAATCATTTT | Exon 55 |
| 2964 | --R--E--N--C--W--K--F--F--M--A--R--V--R--L--Q--L--K--I--I--L | |
| 8949 | GTGTTTCTCTCCAGTTGGTCGCACGCTGAGAGTTAGAGCTCGGAAGTTCCCAGCCATAGT | |
| 2984 | --C--F--S--P--V--G--R--T--L--R--V--R--A--R--K--F--P--A--I--V | |
| 9009 | TAACTGCACGGCTATTGACTGGTTTCATGCGTGGCCGCAGGAGGCTCTGGTCTCCGTCAG | |
| 3004 | --N--C--T--A--I--D--W--F--H--A--W--P--Q--E--A--L--V--S--V--S | |
| | 9113_9116delAAGA[K3038TfsX13] | |
| 9069 | CAGGAGGTTCATTGAGGAAACCAAGGGAATTGAGCCAGTGCACAAAGACTCTATTAGCCT | Exon 56 |
| 3024 | --R--R--F--I--E--E--T--K--G--I--E--P--V--H--K--D--S--I--S--L | |
| 9129 | TTTCATGGCACATGTTCACACCACTGTAAATGAAATGAGTACCAGATATTACCAGAATGA | |
| 3044 | --F--M--A--H--V--H--T--T--V--N--E--M--S--T--R--Y--Y--Q--N--E | |
| 9189 | GAGAAGACACAACTATACCACCCCAAAGAGTTTTCTAGAACAAATATCACTGTTTAAGAA | |
| 3064 | --R--R--H--N--Y--T--T--P--K--S--F--L--E--Q--I--S--L--F--K--N | |
| 9249 | CCTGTTGAAGAAGAAGCAAAATGAGGTATCCGAGAAAAAAGAACGCCTGGTGAACGGCAT | |
| 3084 | --L--L--K--K--K--Q--N--E--V--S--E--K--K--E--R--L--V--N--G--I | |
| 9309 | CCAAAAGCTAAAAACCACAGCCTCTCAGGTGGGAGATCTAAAAGCCAGACTTGCCTCTCA | Exon 57 |
| 3104 | --Q--K--L--K--T--T--A--S--Q--V--G--D--L--K--A--R--L--A--S--Q | |
| 9369 | AGAAGCCGAGCTGCAACTGAGAAATCATGATGCCGAAGCTCTGATCACAAAGATCGGCCT | |
| 3124 | --E--A--E--L--Q--L--R--N--H--D--A--E--A--L--I--T--K--I--G--L | |
| 9429 | TCAGACGGAGAAAGTGAGCCGGGAAAAGACCATCGCTGATGCTGAGGAGCGAAAGGTGAC | Exon 58 |
| 3144 | --Q--T--E--K--V--S--R--E--K--T--I--A--D--A--E--E--R--K--V--T | |
| 9489 | AGCCATTCAGACTGAAGTGTTCCAGAAACAGAGAGAATGTGAAGCTGACTTACTCAAGGC | |
| 3164 | --A--I--Q--T--E--V--F--Q--K--Q--R--E--C--E--A--D--L--L--K--A | |
| 9549 | TGAGCCTGCACTTGTGGCTGCTACAGCTGCACTCAATACACTCAACAGGGTCAACCTCAG | Exon 59 |
| 3184 | --E--P--A--L--V--A--A--T--A--A--L--N--T--L--N--R--V--N--L--S | |
| 9609 | TGAGCTGAAAGCCTTTCCCAACCCTCCCATCGCAGTTACCAATGTTACTGCAGCCGTGAT | |
| 3204 | --E--L--K--A--F--P--N--P--P--I--A--V--T--N--V--T--A--A--V--M | |
| 9669 | GGTCCTTCTGGCTCCTCGGGGAAGAGTGCCCAAAGACCGAAGTTGGAAAGCAGCTAAAGT | |
| 3224 | --V--L--L--A--P--R--G--R--V--P--K--D--R--S--W--K--A--A--K--V | |
| | 9764T > C[L3255S] | |
| 9729 | CTTCATGGGAAAGGTTGATGATTTTTTGCAAGCATTAATTAACTATGACAAAGAGCACAT | Exon 60 |
| 3244 | --F--M--G--K--V--D--D--F--L--Q--A--L--I--N--Y--D--K--E--H--I | |
| 9789 | TCCAGAGAACTGTCTAAAAGTGGTGAATGAACACTATTTGAAAGACCCAGAGTTTAATCC | |
| 3264 | --P--E--N--C--L--K--V--V--N--E--H--Y--L--K--D--P--E--F--N--P | |
| 9849 | AAACCTGATTCGAACCAAATCTTTTGCAGCAGCTGGCCTGTGTGCCTGGGTCATCAACAT | |
| 3284 | --N--L--I--R--T--K--S--F--A--A--A--G--L--C--A--W--V--I--N--I | |
| 9909 | CATTAAATTCTATGAGGTCTACTGTGATGTGGAGCCAAAACGCCAAGCATTAGCCCAAGC | Exon 61 |
| 3304 | --I--K--F--Y--E--V--Y--C--D--V--E--P--K--R--Q--A--L--A--Q--A | |
| 9969 | AAACTTAGAACTGGCTGCAGCTACTGAAAAACTAGAGGCTATCAGGAAAAAGCTTGTGGA | Exon 62 |
| 3324 | --N--L--E--L--A--A--A--T--E--K--L--E--A--I--R--K--K--L--V--D | |
| 10029 | TCTGGATCGAAATCTGAGCAGACTCACGGCTTCATTTGAAAAAGCAACAGCTGAGAAAGT | |
| 3344 | --L--D--R--N--L--S--R--L--T--A--S--F--E--K--A--T--A--E--K--V | |
| 10089 | CCGGTGTCAAGAAGAGGTGAACCAAACCAACAAAACCATCAAATTAGCTAACAGACTTGT | |
| 3364 | --R--C--Q--E--E--V--N--Q--T--N--K--T--I--K--L--A--N--R--L--V | |
| 10149 | CAAGGAACTTGAGGCAAAGAAGATTCGCTGGGGTCAATCCATTAAGTCCTTTGAAGCTCA | Exon 63 |
| 3384 | --K--E--L--E--A--K--K--I--R--W--G--Q--S--I--K--S--F--E--A--Q | |
| 10209 | AGAGAAGACACTCTGTGGAGATGTTCTTCTCACGGCGGCATTTGTGTCTTACGTCGGACC | |
| 3404 | --E--K--T--L--C--G--D--V--L--L--T--A--A--F--V--S--Y--V--G--P | |
| | 10324C > T[Q3442X] | |
| 10269 | CTTCACAAGGCAGTATCGCCAGGAGCTGGTGCACTGCAAGTGGGTTCCCTTTCTTCAACA | |
| 3424 | --F--T--R--Q--Y--R--Q--E--L--V--H--C--K--W--V--P--F--L--Q--Q | |
| 10329 | GAAGGTTTCCATTCCACTAACCGAAGGCCTGGACTTGATATCCATGTTGACGGATGATGC | Exon 64 |
| 3444 | --K--V--S--I--P--L--T--E--G--L--D--L--I--S--M--L--T--D--D--A | |
| 10389 | TACAATTGCCGCCTGGAATAACGAAGGACTGCCCAGTGACAGAATGTCCACCGAAAATGC | |
| 3464 | --T--I--A--A--W--N--N--E--G--L--P--S--D--R--M--S--T--E--N--A | |
| 10449 | CGCTATCCTAACACACTGTGAGCGCTGGCCTCTGGTGATAGATCCCCAGCAACAGGGAAT | |
| 3484 | --A--I--L--T--H--C--E--R--W--P--L--V--I--D--P--Q--Q--Q--G--I | |
| 10509 | TAAGTGGATCAAGAATAAGTATGGAATGGACCTGAAAGTCACACATTTGGGCCAGAAAGG | |
| 3504 | --K--W--I--K--N--K--Y--G--M--D--L--K--V--T--H--L--G--Q--K--G | |
| 10569 | GTTTTTGAATGCCATTGAAACTGCTTTGGCCTTTGGTGATGTCATCTTAATTGAAAATCT | Exon 65 |
| 3524 | --F--L--N--A--I--E--T--A--L--A--F--G--D--V--I--L--I--E--N--L | |
| 10629 | CGAGGAAACGATAGATCCAGTCCTGGATCCACTACTTGGCAGGAACACAATTAAAAAAGG | |
| 3544 | --E--E--T--I--D--P--V--L--D--P--L--L--G--R--N--T--I--K--K--G | |
| 10689 | AAAGTATATCAGGATTGGAGATAAAGAATGTGAATTTAACAAGAACTTTCGCCTTATCCT | Exon 66 |
| 3564 | K--Y--I--R--I--G--D--K--E--C--E--F--N--K--N--F--R--L--I--L | |
| 10749 | TCACACAAAATTGGCAAATCCTCACTATAAGCCGGAATTACAAGCTCAGACAACTCTCCT | |
| 3584 | --H--T--K--L--A--N--P--H--Y--K--P--E--L--Q--A--Q--T--T--L--L | |
| 10809 | CAATTTCACAGTCACAGAAGATGGTCTAGAAGCCCAGCTGCTGGCAGAGGTTGTCAGTAT | |
| 3604 | --N--F--T--V--T--E--D--G--L--E--A--Q--L--L--A--E--V--V--S--I | |
| 10869 | TGAAAGGCCAGATTTGGAGAAACTTAAGTTGGTATTGACAAAGCACCAAAATGATTTTAA | Exon 67 |
| 3624 | --E--R--P--D--L--E--K--L--K--L--V--L--T--K--H--Q--N--D--F--K | |
| 10929 | AATTGAGCTCAAGTATCTGGAAGACGATCTCCTTTTGCGCCTTTCTGCGGCAGAGGGAAG | |
| 3644 | --I--E--L--K--Y--L--E--D--D--L--L--L--R--L--S--A--A--E--G--S | |
| 10989 | CTTTCTGGATGACACCAAACTGGTAGAGAGATTGGAGGCAACAAAGACCACCGTGGCAGA | |
| 3664 | --F--L--D--D--T--K--L--V--E--R--L--E--A--T--K--T--T--V--A--E | |
| 11049 | GATAGAGCACAAGGTGATTGAAGCCAAAGAAAATGAAAGAAAAATCAACGAGGCCCGAGA | Exon 68 |
| 3684 | --I--E--H--K--V--I--E--A--K--E--N--E--R--K--I--N--E--A--R--E | |
| 11109 | ATGTTACAGACCAGTGGCAGCAAGAGCATCTCTTCTTTATTTTGTTATTAATGACCTCCA | |
| 3704 | --C--Y--R--P--V--A--A--R--A--S--L--L--Y--F--V--I--N--D--L--Q | |
| 11169 | AAAAATCAACCCCCTCTACCAATTCTCTTTGAAGGCTTTTAACGTGCTGTTCCACAGAGC | Exon 69 |
| 3724 | --K--I--N--P--L--Y--Q--F--S--L--K--A--F--N--V--L--F--H--R--A | |
| 11229 | GATCGAGCAGGCTGACAAGGTGGAAGACATGCAGGGACGCATCTCTATCCTGATGGAGAG | |
| 3744 | --I--E--Q--A--D--K--V--E--D--M--Q--G--R--I--S--I--L--M--E--S | |
| 11289 | CATCACCCATGCTGTCTTCCTCTACACCAGCCAGGCGCTGTTTGAGAAGGACAAGCTCAC | |
| 3764 | --I--T--H--A--V--F--L--Y--T--S--Q--A--L--F--E--K--D--K--L--T | |
| 11349 | CTTCCTGTCCCAGATGGCTTTTCAGATTTTGTTGAGAAAGAAAGAGATAGACCCTCTTGA | Exon 70 |
| 3784 | --F--L--S--Q--M--A--F--Q--I--L--L--R--K--K--E--I--D--P--L--E | |
| 11409 | ATTGGATTTCCTGCTTCGATTCACAGTTGAACACACTCATCTGAGTCCCGTTGACTTCCT | |
| 3804 | --L--D--F--L--L--R--F--T--V--E--H--T--H--L--S--P--V--D--F--L | |
| 11469 | AACTTCTCAGTCATGGAGTGCTATCAAGGCAATTGCCGTCATGGAAGAATTTCGAGGCAT | Exon 71 |
| 3824 | --T--S--Q--S--W--S--A--I--K--A--I--A--V--M--E--E--F--R--G--I | |
| 11529 | AGACCGAGATGTGGAAGGATCTGCCAAGCAGTGGAGGAAGTGGGTAGAATCCGAGTGTCC | |
| 3844 | --D--R--D--V--E--G--S--A--K--Q--W--R--K--W--V--E--S--E--C--P | |
| 11589 | AGAAAAAGAAAAATTACCTCAAGAATGGAAGAAGAAAAGTTTAATACAGAAGCTGATTCT | |
| 3864 | --E--K--E--K--L--P--Q--E--W--K--K--K--S--L--I--Q--K--L--I--L | |
| | 11663G > A[R3888H] | |
| 11649 | TCTGAGAGCAATGCGCCCTGACAGAATGACGTATGCTCTCAGAAATTTTGTAGAGGAAAA | Exon 72 |
| 3884 | --L--R--A--M--R--P--D--R--M--T--Y--A--L--R--N--F--V--E--E--K | |
| 11709 | ACTGGGTGCGAAGTATGTGGAGAGGACCAGATTGGACTTAGTTAAAGCATTCGAAGAAAG | |
| 3904 | --L--G--A--K--Y--V--E--R--T--R--L--D--L--V--K--A--F--E--E--S | |
| | 11804C > T[P3935L] | |
| 11769 | CAGCCCAGCCACCCCCATATTCTTCATCCTGTCTCCGGGGGTAGATGCCCTTAAAGACCT | |
| 3924 | --S--P--A--T--P--I--F--F--I--L--S--P--G--V--D--A--L--K--D--L | |
| 11829 | GGAGATTCTTGGCAAAAGACTTGGCTTTACAATTGACTCTGGAAAATTCCACAATGTGTC | Exon 73 |
| 3944 | --E--I--L--G--K--R--L--G--F--T--I--D--S--G--K--F--H--N--V--S | |
| | 11929G > T[E3977X] | |
| 11889 | TTTAGGACAAGGTCAGGAGACGGTGGCAGAAGTGGCCCTGGAGAAAGCTTCCAAAGGAGG | |
| 3964 | --L--G--Q--G--Q--E--T--V--A--E--V--A--L--E--K--A--S--K--G--G | |
| 11949 | ACACTGGGTCATCCTCCAAAATGTTCATTTGGTAGCCAAGTGGCTAGGAACCTTGGAGAA | Exon 74 |
| 3984 | --H--W--V--I--L--Q--N--V--H--L--V--A--K--W--L--G--T--L--E--K | |
| | 12064G > C[A4022P] | |
| 12009 | GCTCCTTGAAAGATTCAGCCAAGGAAGCCACAGAGATTACAGGGTTTTCATGAGTGCTGA | |
| 4004 | --L--L--E--R--F--S--Q--G--S--H--R--D--Y--R--V--F--M--S--A--E | |
| 12069 | GTCTGCACCTACACCAGATGAGCATATCATCCCTCAAGGACTCCTGGAAAATTCCATTAA | |
| 4024 | --S--A--P--T--P--D--E--H--I--I--P--Q--G--L--L--E--N--S--I--K | |
| 12129 | GATCACTAATGAACCCCCAACAGGGATGCTGGCCAATTTGCATGCCGCCCTGTACAACTT | |
| 4044 | --I--T--N--E--P--P--T--G--M--L--A--N--L--H--A--A--L--Y--N--F | |
| 12189 | TGATCAGGATACACTTGAAATATGCTCCAAGGAGCAGGAGTTTAAAAGCATCCTTTTTTC | Exon 75 |
| 4064 | --D--Q--D--T--L--E--I--C--S--K--E--Q--E--F--K--S--I--L--F--S | |
| 12249 | TCTCTGCTACTTCCACGCCTGTGTTGCTGGGAGACTGAGGTTTGGCCCCCAGGGCTGGAG | |
| 4084 | --L--C--Y--F--H--A--C--V--A--G--R--L--R--F--G--P--Q--G--W--S | |
| 12309 | CCGAAGCTATCCTTTTAATCCTGGAGACCTCACCATTTGTGCCAGTGTCCTCTACAACTA | |
| 4104 | --R--S--Y--P--F--N--P--G--D--L--T--I--C--A--S--V--L--Y--N--Y | |
| 12369 | CTTAGAGGCAAACTCTAAAGTCCCATGGGAAGATCTCCGTTATCTCTTTGGTGAGATCAT | Exon 76 |
| 4124 | --L--E--A--N--S--K--V--P--W--E--D--L--R--Y--L--F--G--E--I--M | |
| 12429 | GTATGGAGGCCACATCACAGATGACTGGGATCGCAAACTGTGTCGGGTGTATTTAGAAGA | |
| 4144 | --Y--G--G--H--I--T--D--D--W--D--R--K--L--C--R--V--Y--L--E--E | |
| 12489 | ATTCATGAATCCATCTCTGACTGAAGATGAACTGATGCTGGCACCAGGTTTTGCTGCCCC | Exon 77 |
| 4164 | --F--M--N--P--S--L--T--E--D--E--L--M--L--A--P--G--F--A--A--P | |
| 12549 | ACCCTACCTAGATTATGCAGGCTACCACCAGTACATAGAGGAGATGCTTCCTCCAGAAAG | |
| 4184 | --P--Y--L--D--Y--A--G--Y--H--Q--Y--I--E--E--M--L--P--P--E--S | |
| 12609 | CCCGGCACTGTATGGCCTCCACCCAAATGCTGAAATAGAATTCCTGACAGTGACATCCAA | |
| 4204 | --P--A--L--Y--G--L--H--P--N--A--E--I--E--F--L--T--V--T--S--N | |
| | 12697C > T[Q4233X] | |
| 12669 | CACTCTCTTCAGAACTTTGCTGGAGATGCAGCCCAGGAATGCACTCAGTGGTGATGAACT | |
| 4224 | --T--L--F--R--T--L--L--E--M--Q--P--R--N--A--L--S--G--D--E--L | |
| 12729 | GGGGCAGTCTACAGAAGAAAAGGTTAAGAATGTCTTGGATGACATTTTGGAGAAACTTCC | Exon 78 |
| 4244 | --G--Q--S--T--E--E--K--V--K--N--V--L--D--D--I--L--E--K--L--P | |
| 12789 | AGAAGAGTTCAACATGGCAGAGATAATGCAAAAAAATTCAAATAGAAGCCCATATGTTCT | |
| 4264 | --E--E--F--N--M--A--E--I--M--Q--K--N--S--N--R--S--P--Y--V--L | |
| 12849 | TGTTTGCTTCCAAGAATGTGAGAGGATGAATATTCTCATTCGGGAAATACGTATATCACT | |
| 4284 | --V--C--F--Q--E--C--E--R--M--N--I--L--I--R--E--I--R--I--S--L | |
| 12909 | TGAACAACTGGACCTTAGTTTGAAGGGGGAATTGGCATTATCTCCTGCTGTGGAAGCCCA | Exon 79 |
| 4304 | --E--Q--L--D--L--S--L--K--G--E--L--A--L--S--P--A--V--E--A--Q | |
| | 12980T > C+L4327S+ | |
| 12969 | GCAGTTTGCATTGAGTTATGACACGGTACCAGACACTTGGAGCAAACTGGCTTATCCTTC | |
| 4324 | --Q--F--A--L--S--Y--D--T--V--P--D--T--W--S--K--L--A--Y--P--S | |
| | 13061T > A[L4354H] 13065_13067delCCT[4356delL] 13075C > T[R4359X] | |
| 13029 | TACTTATGGCCTAGCCCAGTGGTTCAATGACCTCCTCCTGCGATGCCGAGAACTCGATAC | Exon 80 |
| 4344 | --T--Y--G--L--A--Q--W--F--N--D--L--L--L--R--C--R--E--L--D--T | |
| 13089 | TTGGACACAAGACCTTACCCTTCCGGCTGTCGTGTGGCTCTCCGGCTTCTTCAACCCTCA | |
| 4364 | --W--T--Q--D--L--T--L--P--A--V--V--W--L--S--G--F--F--N--P--Q | |
| 13149 | GTCCTTCTTAACTGCAATCATGCAGACGATGGCTCGAAAAAATGAGTGGCCCCTGGATAA | Exon 81 |
| 4384 | --S--F--L--T--A--I--M--Q--T--M--A--R--K--N--E--W--P--L--D--K | |
| | 13213de1C[R4405AfsX1] | |
| 13209 | AACGCGCTTGACTGCTGATGTTACCAAAAAAACAAAGGAAGATTATGGACACCCCCCAAG | |
| 4404 | --T--R--L--T--A--D--V--T--K--K--T--K--E--D--Y--G--H--P--P--R | |
| 13269 | GGAAGGTGCATACCTCCACGGACTCTTCATGGAGGGCGCCCGCTGGGACACCCAAGCAGG | Exon 82 |
| 4424 | --E--G--A--Y--L--H--G--L--F--M--E--G--A--R--W--D--T--Q--A--G | |
| | 13333_13334insACCA[I4445NfsX3] 13373C > T[P4458L] | |
| 13329 | AACCATTGTTGAAGCCCGTCTCAAGGAGCTGGCATGCCCTATGCCGGTCATCTTTGCAAA | |
| 4444 | --T--I--V--E--A--R--L--K--E--L--A--C--P--M--P--V--I--F--A--K | |
| 13389 | AGCCACCCCCGTGGACAGACAAGAAACCAAACAGACCTACGAGTGCCCTGTGTATAGAAC | |
| 4464 | --A--T--P--V--D--R--Q--E--T--K--Q--T--Y--E--C--P--V--Y--R--T | |
| | 13504_13505insGAAGA[T4502RfsX14] | |
| 13449 | CAAACTGAGAGGCCCCAGCTACATCTGGACCTTCAGGCTGAAGAGCGAAGAGAAGACTGC | |
| 4484 | --K--L--R--G--P--S--Y--I--W--T--F--R--L--K--S--E--E--K--T--A | |
| 13509 | AAAATGGGTTCTGGCTGGAGTGGCTCTGCTTCTAGAAGCGTAAGGTAACACTGGCATTCC | |
| 4504 | --K--W--V--L--A--G--V--A--L--L--L--E--A--*-................ | |
| 13569 | TCTAGCCTCTGCTGGAGTGCAGTGAGGATTTTCTAGCATGTTGCTGCACTGTTCCCATGC | |
| | ............................................................ | |
| 13629 | ACATTATTCTAACTTTTTAGTAACTCACACGTGCATTCTTTTTTCAACGCTATCCTTAGA | |
| | ............................................................ | |
| 13689 | GTGAAAGTCAGAAAAAAATACTAGAAACTAACTCAGGGCTGAGCGTGGTGGCACACGACT | |
| | ............................................................ | |
| 13749 | GTAATCCCAGTTACTCAGGAGGTAGGAGAATCACTTGAACCTAGGAGGCAAAGGTTGCAG | |
| | ............................................................ | |
| 13809 | TGAGCCGAGGTTGCACCACTGCACTCCCTCCTGGGCAACAGAACAAGACTCCATCTCAAA | |
| | ............................................................ | |
| 13869 | AAAAAAAAAGTACATCATAAAAGTACATCATATGTGAACATGCAAAAGCAATGCAGCCGG | |
| | ............................................................ | |
| 13929 | AAAGAACGGAGATTTTAATTTTTAACAAACAACAAATTAAATTATTAGCCCTTAAACTCT | |
| | ............................................................ | |
| 13989 | TTCAAAATATAAAAGCAGCAGGCCCCAGGTGAGTCCTGAAGGAAGAGGCTAGCACTCTGT | |
| | ............................................................ | |
| 14049 | AAGGCCTCCAGTGTCCAGTGTCTACAATGTTGATGGTCCCCTTTTGTTCAGTCAAGTTTT | |
| | ............................................................ | |
| 14109 | AATAAAAATAAAACTGTTCTACAGTTAA | |
| | ............................ | |

INTRON 13 of DNAH11 gene (Ensembl No. ENSG00000105877)with site of IVS13−1G>C PCD mutation in bold

GTTTGTGATTTTTGTTAAAAAAAAGATACTAGGGCCTATTATGAATATAA
ATATTTTGAATTTTAATTATTATATAGATCTATATGATATTTTTACACCT
TTAAGACAGAGAAAATAGCTAACATATTTGGCACTTTTTGTTTTGGGGTT
TTCTTTGCTCAG

INTRON 23 of DNAH11 gene (Ensembl No. ENSG00000105877) with site of IVS23+5G>T PCD mutation in bold.

GTCAGTATCCTTGGTCTCACTAATGAACCTTTTTATGACTGTGAAGTGTT
ACTTCTTGGTTTGCATCATCTGAGATGAAGTGTAATTTATGAAAAGACCC
CCCTCAGCATAGCCTCTACTTGCTGTGTTATGCCTTTTTGCTGTCTAGAA
AAAGTCGCCTGATCCGACCAACAATTTAGAAAAAGGCACCCTCCTCCAGG
CTGTCCCCAGGACAGCAGAGCGGAGGCTGGCTTCCATCTCCATTTGTCAT
CTGGGCTGGTGTCCACGTTCAGTACACAGATTGCACAACTAATCAGGGTG
GCCCTATTACTCATCTCTCCTCATTCTTTCTCAAGGACAGACCTAGCTCC
CTGGCTCCCAGCAACTCTGGAGTGAGGCCAAAGTTGACACAGAGATAGTC
TGGCAAACTCATCTAGCTTTCTCAGGACTTTCCAGATTTTAGCACTGAAA
GTATCACATCCCTGAAATCCACTTGCTAGAAACCCCTCAGTCCGGAGCAA
ATCAGGGCATTTGGTCCTCTTATGCAGAGGCGTGGATGGTGCTAAGGAAT
GTTAGAGGGTAGGGAGGGGCAGAGGGTGGCAGCTTCCAAGAAGCAGATTC
TGGATATTCTTAGCAAAAGCCGGAAAGAAAATGGCTTTCCTGCAACCCAT
ATGGCTGGCTCAACTCTTCACTTACAGGAGACATGAGTGAGTTTGGGTAT
GTCGAAGAGCTGAGGATGTCTCAAGGACCCAATATAGGGGAGGGAAGGAG
AGTCAATGTGAGAGAAGGGGAGTCATTGTCACAAGACAATGTGGAAATGA
GAATTATTTTTCACATGGTAAATATGAATAATGACACTGTAAGACTATTT
TTTTTGCCAATGAACTGAGATTAAATTAGTGATAAAACTAGGTTAAATCA
CAGAAGCAGCTGGGAATTATGTTCAGTGAATAGGGTTCTTTAGGAAGAAA
AGACAAATGAGCCACTACCTGATTCCTGGGATTTCAACCAAATTATAATA
AATGAACAATGAGATTCTCTTTAAAAATGGGTTTTGTGGGCAAAGGAGAT
AAGAAACGTGCCGACAAGGGAAAGATGTGAAACAAAGAAAATAGTGTAGC
ACGGATGTGACTAAACTGTGGCCCTTGCTTCCGGCAGGTGACGCCTCAGG
AATTGTGAGTTAAAAAGGAGTGTCTTAGATTCTCCACGTATTTCAAGACG
AGAGATGTACTCAGCACCTGACTTGAGTATATTTTAGTTAAGATTCATTT
CACCAGCCTTTAGGCAAAAGGAACCGTTCATATATGTGGAATATAAAGTC
TAACTTTTTTTCCCCCAATCAG

INTRON 26 of DNAH11 gene (Ebsembl No. ENSG00000105877 with site of IVS26−1G>A PCD mutation in bold.

GTTTGTCAAAGACAGGCTGTATGCTATTCTAGCAAAGTTTTGTAAAGTAA
CATGGTTTTGAGCATCGTATTTATAAAAATGAATGCATTTTTGCATGAGT
AAGCAGGAGTCAGGAGACTTTTCCTGCAAAGGGCCAGGTAATAAATATTT
CAGGCTGTGTGGATCATGCAGCCTGTCTAACACCTTCTGAATGCTGCCAT
TACAACATGAAAGAAGACATGGGTAATAAATAAACAAATGGATATGGTTA
TATGCCAATAAAGCTTTACTTGTAGACACTGAAATTTGAATTTCACATAG
TTATCATGTGTCACAAAATACACTGTTTTTTATTTTTTTCACTGATTTAA
AAAGGTAGGCTAGGCTCGGTGGCTCACACCTATAATCCCAGCCTTTTGGG
AGTCCAAGGTAGGAGGATGACTTGAGGCCAGGAGTTCGAGACAAGCCTAG
GCAGTATGGTGAGATCCCTTCTCTACTAAAAAATTTTTAATAATTGGCTA
GGCAAGGTGGCCCAAGCTTGCAGTCCTAGCTACTTGGGAGGCTGAGGTGG
GAGGATCACTTGAGCCAAGGTAGTTTGAGGTTGCAGTGAGCTATGACTGC
AGCACTGCACTCCAGCCTGGGTGACAGAATGAGACCCTGTCTCCAAAATA
AATTTAAAAACCATTTTTAGCTGCTAGGTCGTGCAAAAACAAACATCAGG
CCATATTTGTCCCATGCACCATAGATTGCCGACCTTTGTGGTAATAAAGG
TAAACAGATTCATCTTCAGAAGTAAAAGACTGAGAAAGGACAACAGGTAG
AAAATAACACTACTAGTTTCGGTTTTTCTTTTCTGTAAAAGCCATAGAAA
GCACGTGGCATGGTGCATGTAATGATAATTGATTGGAGAGATTTCTAGCA
AAGGAGAGTGACTATTTCTCATAAAAATTGAGATTAAAAGGTTTTTTCTT
TCTACCTGAAGTAGAAGTCACAGATAAATTTTAAGTCATTTAACTTTTGA
GGAAGAAAAAATAGATTGTTTATATCTCTCTCATTTTTATATGTAGGTAT
GCTTTTTAAGATTCTGGGCGCTCTCTCATTCTCTGTCTCTCTCTTTCTCT
CTCTCCCTTCCTCTCTTTCTCCCTTTCTCTCTCCTTCTCCTCCCCCTCTG
CCCCCACCTCCTCCTACTTGTCTCCCTTTCTCTTCTTTTTCCTCTTCCCA
TTCTCCCTTCCTTGTCTCCTTTTGCCTTTATTCTTTTTCGTTTGATTTAG
AAGAGAAAAATTCTGTGTGGGCCTAAAGTGGTGACAGTGCTTATCACATT
TATTTTTATAAAAACGGTGTGCTTCTACATTGCTTTTGATCTTCATCTAA
GAGTTGAAAAGCAAAAGTAAACAGATGTGGTGTCAGACATTCTTAGAAGT
ATCTTTGACCTTGCCTCTTCATTCTTTGTTCTTCATTTTTTTAATTCTTG
AAATGATTAAAAGTTTAGAAAAGATTGTTCTAATATCCACGGCCCCGTAT
TGTACTTTCATGCAG

INTRON 34 of DNAH11 gene (Ensembl No. ENSG00000105877) with site of IVS34+1G>A (renumbered as IVS33+1G>A in updated sequence) PCD mutation in bold.

GTAAGTTAGTAAGAGAATAATGTGTAAAACTTTATTCTCTAACATTATTC
CTGATTGGGAATTATTCAATATAATAGCGTAAAAACCCCTTCTGTTAAAT
TCTGAGTGCCTCACTTTATCATTTAG

INTRON 45 of DNAH11 gene (Ensembl No. ENSG00000105877) with site of IVS45+1G>A (renumbered IVS44+1G>A in updated sequence) PCD mutation in bold.

GTATGTTTAGAAATAGTTTACAGGACCAGTTTCCAGTTTTGTGTGGGACA
GGGTCATGGGGAGGTTAAAACATGTGATCTGTACCTTTTTGTTATGTTAT
AGATCTATACTGCTTGCCCTGTTCTCTCCTAAGTCTATGCATTCTCTGGG
TAGTCCCATCCCTGATGGTTCTAACCCCAGCTGACAATGCCAAATCCATA
CCCTCCAGCCTGGCTGTGTCTTCTGAACTGAAAGTTCTGTGTTTCTGGCT
GCTCTGGGGCCTCGCCTTCAGCATGCTGTTCTACAAGAAGCCCCACTCAG
TGTGCCTGAGACAGATCTCCTCCTCTGCCCCATACTCCAACTTGCCCCTG
CTCTTCCTTCTTTTCTTTTGCTCAGTGGTACATTCTTCCCGGTAGCTCCA
GTTAAAAACGTGGAAGACATCACTCTTCCTGATTTCTGTGTCCTGTGATC
TCAGACCCTTTCAGTTTCACCTGTGAAATAGCTGCCAACCCTATTCCCTC
TTCTCCATCCCTGCAGCCTAAATTCAGGAGCAAAGCATCTTTAGATTGCT
CTGGTTATCATAGCGTCTTAGTAGCTCAGATCGCCATAACAAAATACCAC
AAATTGGGTGACTTAAGCAATAGAAATTTATTTCTCACAGTTCTGGGGAC
CAAAAGTCCAAGATTATGGTGTCAGCATGGTTGAGTTCCAGCAAGGACTC
TCTTCCCACCTTTCAGACCGCCACTTTCCCGCTGTGTCCCCATGTGACAG
AGAGAGAAAGACATACTGGTTTCTCTTTTAATGAGGACACTAATCCCATC
ATGAGGACCCTGTATTCATGACATCATCTAAATCTAATTATGTCCCAAGG
CCCCACCACCAAATATCATCACACTGTGGGGTAGGGCTTCAAAATATGGA
TTTGGGGGTGGGATGTGGGAGGGGGGATCACAATTCGGTTTGTAGCATTT
GATAATCTTCAAATTGGTCTCCCTCTCTCCAGTCCCTCTTACTTCTAACC
AGTCTCACACACTATTCCCAGGATAGTCATTTGTGAACCATAGTCAACCC
TGGGTCAATTCCTTTTTCACTGATCTTTTTTTTAGCTGAAAGTGAAATAT
TCATGCCTGTACAGATTGCTTGCATAGCCCTTTTGTGTCTTCTGATTTCC
TGTGGGAAAACACTTGAGACAAACATTTGCACCAGCACACAGATCCCACG
AACTGCTTAGACTTACCAAAGTGCAAAGTGGGAATTATTCAACTTTAGAA
ATTTGCAGATCTAATTGCCACCATGCACCTCTATGTGTAATTTTTCTCAT
GTGATCTTTACAATGGTTCTTCAAGGTATCCTCATTGCACAAATAAAGTA
AAGCTCAGAGGGGCAAAGTAACTTGCCCAAAGTCACGCTGATGGGAAATG
GCAACATCAGGATTCCCACCCAAGTGTGTCTTTCTCCAAAAGCCACACCT
TCCATGGTACCTCAGGGCCTCTCATGGTTCATAGTGCTTCTATGGGTACC
CCTGAATTTTTACATTAAAAATAGATAGCACACATTTTCCAGGCTTCCTT
GCTCTTTCCAGGTGTAAATGGGGCATTTACATTGAAAGGCATCGCAGCCA
AGAGTGACCTATATTTCTGTTGGTTTGTTCACTGTAGATCAGAAAATAAA
TGGTTGATATAATCTGTTGGCCAGTTCCACTTCTCGCCACTAGGTTACCA
TAACTCATGCCTATTATTATAATAATATAGTTGGAATTCACTTGGCCTCC
TAGATCTTATTTCAGCCATTCTCAGAAGCCAGATTCCTTCTGTAATTTCC
AAGTGCTTTCTAAAGATTAGCAGCCAATACTGTGTGAGAGTCAAGTGTTC
GTGTCAGGGACTTCATGTGGCTTTCTCAGAGGCACGGTTTACAACATGTG
TGGGTTTCTTGTTAATGGAGGCCACATGCAGACAGAAGGCAACCCTCCTT
CTCCACCAGGTATTTCTTTCAATATTTACCCTTTTCTCCCTTTCTGGGAC
TGTAAAATCAGGTGGGATGTTGATAGCGGACATCTTCCCCCTCCTGCTCA
TAACCCCTCACCTCCCTCCCAGCTGACCCTCTTTTAATCTTTCCAGTTTC
CAAATGTATCAGCCTTACCCCCTAGTTATATAAGCTAGAAGCTGGATATC
ACTGGCACCTTCCCTTCGCTAATTAATAGTCAACATGAGCAGCAATAGCA
GCTTAACATTTTCAAAAGGGCTCTAACTACAGAGCATTTAAAACAGAGGC
TATGAGGTTGGCAATATGCATGAAACAGACACAATAAATGTTCTCAGGAT
AGCCACTTGGTAGCTTAACAAATTGAGTGTCTAATATGTGTCAGGGAATG
TGCTTAGAGCTTTTCATAGGGCACCTCATTTACTCTTCAAAAACTATAAA
ACTCTATATGGATTGGTACAACTCTTCTTTTACAAATAAGGAACTGAAGC
TTAGAAAGGTATTTTAATCTGCTTCCAGGCACAAAGCTAACCCATGTCTA
AGCCAGGTTCAAACAGCAGCGGACTGACTCAGGACCTATGTTCTTCAGCC
ATTGCAGCCAACCTCCAAAACAAGTTTCAAATCAACTTTATCTGAACTCC
CATAAACTGCCGTCATTTCTCACTTGGCTTCTGAAGTAGCCTCCTCACTG
GGCTTTCTATTTCTGCATTTATTCTGATAATTCATCATCCACTCAGCAGT
CAGGGTGATTTTTTGAAATGCACGTGAAATCCTATGATATACTCCAGAGT
TGAAAACACTTGAATGGCTCCTTGTTGCCTTTAAGATTACATTTCAAATT
TTAATTCTATTTACAAGGCCTCTGAATCTAGCCCTGCTTAGCTCTCCCTA
TTTCTTCCTCTGCCACTTCCCCCACTCCCCCACTCCCCCACCCCTCCACC
CCCAGCTACAGGGTCCAGTTTCTTGAGCACACCAAGTTCTTTCATACCAC
AGGAACTTTGTACATGCTGGACTCTTCCTTAAACGGCACTTAAAGATGCC
TTCCCTGAACATCCAATTAAAATAGGTTGCCTCTGTTATTCTCACTTACA
GCAAACTTTACTTTCAAGGCAACTGTTGATGTCTGCGTTTGTTGGCTTAG
CCTCAGCTGGACGACACATCTTCATGTTCCTCTCCTCTATGTTCCCCATA
AACTCTGTGAGAGGAGAGACTCTGTTGGTTTTCTTTTCTACTTTAGAACA
GATGCGTTTTAGTTGAATTAATTAATGAATGAAAGATTTGTCACCAAAAT
CATTTTGCCTATATGTAGTGGTTAGGTGAAGCAAAGTTTGATATCAAGTA
AAAGTTAAATGTAGTCTGCCTGCAGAGAATGGTGTCACATAAGTAATATC
ATCTGAACTGTCCTGGCTCAAGACCAACTTTTGTAGGGTGAAGAAGTATC
TTCTACCACCAATCTGAAAATAATTTAAATGAACCTAAAGTCATAACTCA
TGCATGGGGTGGTAGAAGGATGCTCTCAGCTGGTTTGGAAGATGGTGGGG
CAAGCTCTCCCTGCAAACACCGATCACCTCCTTCTGCCCAGTTTATAGGC
CATAATCCAATGACATAGGGACGTCAAAGTAAAAGCAAGTTTAAAAACTT
CTATGGGGAAAAATCACGGTTGAAGTCATAGATAGAAATGAGATTGCTGA
AAGCAGTAGACACACACAGTATTTTTAAGAGTTCATCAAACTCACTATTT
TAGGTCATGCTATGGATATAGGAGTCACTGCGCCAGGTCTCCTGTGGATA
TAAGAGGTACAGAGCCAGGTCATCGTATGAATATAGGAGTCGCTGAGCCA
GGTCATCCTATGAATATAGGAAACACTGGGCCAGGTCATCTGTGGATATG
GGAGTAACTGGGCCAGGTCATCCTATGAATATAAGAGTCACTGGGCCAGG
TCATCCTATGAATATAGGAGTCACTGGGCCAGGTCATCCTGTGGATGCAT
AGAGGTATCACTGGACCAGGTCATCATGTGGATATGGCAGTGACTGGACC
AGATCATCCTAGGGATATAGGAGTCTCTGGGCCAGATTATCCTATGGATA
TAGGAGTCACTGGGCCAGATCATCCTGTGGACATGGGCATCACTGGGACA
GGTCATCCTGTAGACATGGGCATCACTGGGCCAGGTCATCCTGTGGATAT
AGAAGGGTTTTTCTAGTTGTTGCTCACAGTTGTCCTTTTCTTTATTTTCC
AACCCTAATCTAGCAGTAAATGGCTTATATCTATACTGATATTAAAGCAA
GCACCTCCCAGTAAAGAGAGCCTGTTCCTTGCCAATATAAATCTCTCTGG
AGGCTGAAAGTTGATCCCTATGCAGTTTTCAGCTGGACCCCTTAGATTTG
GTCTGGTGGGAAACCAAGCCCATGCGTTTATCCATTCCTCCATCTCTCAT
GGCAGGGATTCCCAGATTTAGAATTCCAGAGATCAACAGAGTTTCAGTAA
TGATGTGTAGGAATGGTATAGAGTTGCCAGTTGTTTACTTTTCTAAGCAA
GAGCATTTATGAAAATAACTATCACCATAATTTTATAAGGGAAAAATATT
TTATCATTGAAAGGGCCAGAGAAGCTATAAGCTCAGAACAAAGGACAGTA
TTTAAATCAAATAATGTCATTAATTTTTAAAAAGTACGCCATTCTTATTT
TTCCCATTTTCCCATAAATTGATGTGAACATTGGTAGAAGTGAACACCAC
CGTTACAATAGTGACTACCTAGCATAGGAATTTGCATAATACGTTGTCAT
TAGATTGAATAACGAACAGGTAATCTGGCGTTCACCTAACTTTACCTTGC
TTTTTAAGTTAATTATTTCACAAAATTTGTGCTTGATGGGTATATGGCAA
TTTTAGGTTTCTACCCCTATGATATTGTTACTCATAATTTGTTTCTTTTT
TTACAGTTTTAATTACTTTGAGTCTGCAATAAGGATTTCTTTTGTTCTCC
TTAG

That which is claimed is:

1. A method of diagnosing primary ciliary dyskinesia (PCD) in a subject, comprising detecting in the subject at least two mutations in the DNAH11 gene of the subject, wherein the mutations are selected from the group consisting of:
1) 350A>T (E117V);
2) IVS13−1G>C (Y759_E889del)
3) 2569C>T (R857X);
4) 3901G>T (E1301X);
5) IVS23+5G>T (E1366_G1418de1);
6) 4333C>T (R1445X);
7) 4438C>T (R1480X);
8) 4516_{—}4517delCT (L1506fsX10);
9) IVS26−1G>A (E1576AfsX4);
10) IVS33+1G>A (V1821TfsX7);
11) 5815G>A (G1939R);
12) 6244C>T (R2082X);
13) 7148T>C (L2383P);
14) IVS44+1G>A (T2379_Q2422del)
15) 7914G>C (W2604X splice)
16) 9113_{—}9116delAAGA (K3038TfsX13);
17) 9764T>C (L3255S);
18) 10324C>T (Q3442X);
19) 11663G>A (R3888H);
20) 11804C>T (P3935L));
21) 11929G>T (E3977X);
22) 12064G>C (A4022P);
23) 12697C>T (Q4233X);
24) 12980T>C (L4327S);
25) 13061T>A (L4354H);
26) 13065_{—}13067delCCT (4356delL);
27) 13075C>T (R4359X);
28) 13213delC (R4405AfsX1);
29) 13333_{—}13334insACCA (I4445NfsX3);
30) 13504_{—}13505insGAAGA (T4502RfsX14);
31) 13373C>T (P4458L); and
32) any combination of (1)-(31) above.

2. A method of confirming a diagnosis of PCD in a subject, comprising detecting in the subject at least two mutations in the DNAH11 gene of the subject, wherein the mutations are selected from the group consisting of:
1) 350A>T (E117V);
2) IVS13−1G>C (Y759_E889del)
3) 2569C>T (R857X);
4) 3901G>T (E1301X);
5) IVS23+5G>T (E1366_G1418del);
6) 4333C>T (R1445X);
7) 4438C>T (R1480X);
8) 4516_{—}4517delCT (L1506fsX10);
9) IVS26−1G>A (E1576AfsX4);
10) IVS33+1G>A (V1821TfsX7);
11) 5815G>A (G1939R);
12) 6244C>T (R2082X);
13) 7148T>C (L2383P);
14) IVS44+1G>A (T2379_Q2422del)
15) 7914G>C (W2604X splice)
16) 9113_{—}9116delAAGA (K3038TfsX13);
17) 9764T>C (L3255S);
18) 10324C>T (Q3442X);
19) 11663G>A (R3888H);
20) 11804C>T (P3935L));
21) 11929G>T (E3977X);
22) 12064G>C (A4022P);
23) 12697C>T (Q4233X);
24) 12980T>C (L4327S);
25) 13061T>A (L4354H);
26) 13065_{—}13067delCCT (4356delL);
27) 13075C>T (R4359X);
28) 13213delC (R4405AfsX1);
29) 13333_{—}13334insACCA (I4445NfsX3);
30) 13504_{—}13505insGAAGA (T4502RfsX14);
31) 13373C>T (P4458L); and
32) any combination of (1)-(31) above.

3. A method of identifying a subject as having an increased likelihood of having PCD, comprising detecting at least two mutations in the DNAH11 gene of the subject, wherein the mutations are selected from the group consisting of:
1) 350A>T (E117V);
2) IVS13−1G>C (Y759_E889del)
3) 2569C>T (R857X);
4) 3901G>T (E1301X);
5) IVS23+5G>T (E1366_G1418de1);
6) 4333C>T (R1445X);
7) 4438C>T (R1480X);
8) 4516_{—}4517delCT (L1506fsX10);
9) IVS26−1G>A (E1576AfsX4);
10) IVS33+1G>A (V1821TfsX7);
11) 5815G>A (G1939R);
12) 6244C>T (R2082X);
13) 7148T>C (L2383P);
14) IVS44+1G>A (T2379_Q2422del)
15) 7914G>C (W2604X splice)
16) 9113_{—}9116delAAGA (K3038TfsX13);
17) 9764T>C (L3255S);
18) 10324C>T (Q3442X);
19) 11663G>A (R3888H);
20) 11804C>T (P3935L));
21) 11929G>T (E3977X);
22) 12064G>C (A4022P);
23) 12697C>T (Q4233X);
24) 12980T>C (L4327S);
25) 13061T>A (L4354H);
26) 13065_{—}13067delCCT (4356delL);
27) 13075C>T (R4359X);
28) 13213delC (R4405AfsX1);
29) 13333_{—}13334insACCA (I4445NfsX3);
30) 13504_{—}13505insGAAGA (T4502RfsX14);
31) 13373C>T (P4458L); and
32) any combination of (1)-(31) above.

4. A method of identifying a carrier of a PCD mutation or identifying a subject having an increased likelihood of having PCD, comprising detecting in the subject a mutation in the DNAH11 gene of the subject, wherein the mutation is selected from the group consisting of:
1) 350A>T (E117V);
2) IVS13−1G>C (Y759_E889del)
3) 2569C>T (R857X);
4) 3901G>T (E1301X);
5) IVS23+5G>T (E1366_G1418del);
6) 4333C>T (R1445X);
7) 4438C>T (R1480X);
8) 4516_{—}4517delCT (L1506fsX10);
9) IVS26−1G>A (E1576AfsX4);
10) IVS33+1G>A (V1821TfsX7);
11) 5815G>A (G1939R);
12) 6244C>T (R2082X);
13) 7148T>C (L2383P);
14) IVS44+1G>A (T2379_Q2422del)
15) 7914G>C (W2604X splice)
16) 9113_{—}9116delAAGA (K3038TfsX13);
17) 9764T>C (L3255S);
18) 10324C>T (Q3442X);
19) 11663G>A (R3888H);
20) 11804C>T (P3935L));
21) 11929G>T (E3977X);
22) 12064G>C (A4022P);
23) 12697C>T (Q4233X);
24) 12980T>C (L4327S);
25) 13061T>A (L4354H);
26) 13065_{—}13067delCCT (4356delL);
27) 13075C>T (R4359X);
28) 13213delC (R4405AfsX1);
29) 13333_{—}13334insACCA (I4445NfsX3);
30) 13504_{—}13505insGAAGA (T4502RfsX14);
31) 13373C>T (P4458L); and
32) any combination of (1)-(31) above.

5. The method of claim 1, wherein the subject does not have PCD-associated dynein arm ultrastructure as analyzed by electron microscopy.

6. The method of claim 1, wherein said detecting comprises performing a hybridization assay.

7. The method of claim 1, wherein said detecting comprises performing a nucleic acid amplification assay.

8. The method of claim 1, wherein said detecting comprises sequencing nucleic acid of the subject.

9. The method of claim 1, wherein said detecting comprises performing a restriction fragment length polymorphism analysis.

10. The method of claim 1, wherein said detecting comprises performing a high performance liquid chromatography analysis.

11. The method of claim 1, wherein said detecting comprises performing a ligase chain reaction assay.

12. The method of claim 1, wherein the subject has a family history of PCD.

13. A kit comprising reagents to detect one or more mutation in a DNAH11 gene, wherein the mutation is selected from the group consisting of:
1) 350A>T (E117V);
2) IVS13−1G>C (Y759_E889del)
3) 2569C>T (R857X);
4) 3901G>T (E1301X);
5) IVS23+5G>T (E1366_G1418del);
6) 4333C>T (R1445X);
7) 4438C>T (R1480X);
8) 4516_{—}4517delCT (L1506fsX10);
9) IVS26−1G>A (E1576AfsX4);
10) IVS33+1G>A (V1821TfsX7);
11) 5815G>A (G1939R);
12) 6244C>T (R2082X);
13) 7148T>C (L2383P);
14) IVS44+1G>A (T2379_Q2422del)
15) 7914G>C (W2604X splice)
16) 9113_{—}9116delAAGA (K3038TfsX13);
17) 9764T>C (L3255S);
18) 10324C>T (Q3442X);
19) 11663G>A (R3888H);
20) 11804C>T (P3935L));
21) 11929G>T (E3977X);
22) 12064G>C (A4022P);
23) 12697C>T (Q4233X);
24) 12980T>C (L4327S);
25) 13061T>A (L4354H);
26) 13065_{—}13067delCCT (4356delL);
27) 13075C>T (R4359X);
28) 13213delC (R4405AfsX1);
29) 13333_{—}13334insACCA (I4445NfsX3);
30) 13504_{—}13505insGAAGA (T4502RfsX14);
31) 13373C>T (P4458L); and
32) any combination of (1)-(31) above.

14. The kit of claim 13, wherein the reagents comprise oligonucleotide primers to amplify a nucleotide sequence of the DNAH11 gene in one or more regions comprising a PCD mutation.

15. A computer-assisted method of identifying a proposed therapy and/or treatment for PCD as an effective and/or appropriate therapy and/or treatment for a subject that has PCD, comprising the steps of:
(a) storing a database of biological data for a plurality of subjects, the biological data that is being stored including for each of said plurality of subjects:
(i) therapy and/or treatment type,
(ii) at least one PCD mutation, and,
(iii) at least one disease progression measure and/or symptom for PCD from which treatment and/or therapy efficacy can be determined; and then
(b) querying the database to determine the dependence on said PCD mutation(s) of the effectiveness of a treatment and/or therapy type in treating and/or managing PCD, thereby identifying a proposed treatment and/or therapy as an effective and/or appropriate treatment and/or therapy for a subject with PCD.


SEQUENCE LISTING (7 sequences)

SEQ ID NO: 1 - DNA, 14167 bp, Homo sapiens
  CDS (32)..(13582)
ccggcctcgcgttccctcggacggttgcccaatggcagcccaggtggcagcccgggaggcgcgagacttcagagaagccccgacccttcgcctaacctcgggggccggcctggaggcagtgggcgctgtggagctcgaggaggaggaggagaacgaggaggaggcggcggccaggagagcgcggagtttcgcccaagacgcgcgggtgcgcttcctcggcggccgcctggcgatgatgctggggttcacggaggagaaatggagccagtatttggaaagcgaggacaaccggcaggttcttggggagtttctggaaagcaccagcccggcttgccttgtgtttagcttcgccgcctcggggcgccttgcggcttcccaggagattccaagagatgcaaaccataaacttgtttttatttccaagaagattactgaaagcattggagtaaatgacttttctcaagtggttttatttggagagttacctgcgttgtctcttggacatgtatctgctttccttgatgagattttagtgccagttctttctaataagaacaaccataagtcctggtcctgttttacttcacaagatatggaatatcacatagaagtcatgaaaaagaagatgtatatttttaggggcaaaatgtctagaagaactcttctaccaattcccactgttgcaggaaagatggatctggatcagaattgttcagagaacaagccaccgtcaaacgaaaggataatacttcatgcaattgaatctgtggttattgaatggtcacatcaaatccaagaaattatagaaagagattcagtgcagcgtttgttgaatggtcttcacttgtctcctcaagcagaactagatttctggatgatgaggagagaaaatctgtcatgcatttatgatcaacttcaggcacctgttgtcctcaaaatggttaagatcctgacaactaaacaaagcagctattttcctactctgaaggacatttttctggctgtggaaaatgctcttctcgaagcccaagatgtggaactttacctgagacctctgaggagacacatccagtgtctccaggagacggaattcccacagacacgcatattaatcgctccattatttcataccatctgtctgatctggagtcattccaagttttataacaccccagctcgggttatagttttattgcaagagttttgtaatctcttcattaaccaggcaacagcttacctttcacctgaggaccttttgaggggagaaatagaagagtcactggaaaaggtgcaggtggctgttaacatcttaaagactttcaaaaactcctttttcaactatagaaaaaaattggcaagctactttatgggaagaaagctgagaccatgggatttccagtctcatctggtgttttgcagatttgacaagtttcttgatcgtttaataaaaatagaggatatatttgccaccactttggaatttgaaaagctggaaagactggaatttggtggtaccaaaggagcaattttaaatggacaagtccacgagatgagtgaagaacttatggaactctgtaaactttttaaacagagcacttatgacccatctgattgcactaacatggagtttgaaagtgattatgtggcatttaagtccaaaactctggaatttgacagaaggcttgggacaattatttgtgaagctttctttaactgcaatggcttagaagctgcatttaagcttttgaccatatttggaaattttctagagaagccagttgtcatggaaattttcagcctacattacagcacactagtgcatatgtttaatacagagctggatgtgtgtaagcaactgtataatgaacacatgaaacagattgaatgtggtcatgtagttcttaacaagaacatgccatttacctcaggaaatatgaaatgggcccagcaggttctccaacgacttcaaatgttttggtcaaacttcgcatctctccgttatctatttttgggcaatcctgatcacgctttagtttatcaaaagtatgttgaaatgaccactttgcttgatcaatttgaaagtcgtatctataatgaatggaaaagtaatgtggatgaaatctgtgaattcaatttgaatcaacccttggttaaattcagtgccataaatggtcttctctgtgtcaattttgacccaaagctagtggctgtattgagagaagtgaaatatcttttgatgttgaagaaacaagacataccagattcagctttagccatcttcaagaaaaggaacactattttaaagtacattggaaatcttgaccttcttgtgcaagggtataataaactcaaacagacgctcctggaagttgaataccctctgattgaagatgagctgagggctattgacgagcagctgacagcagccacaacgtggctgacatggcaggatgactgctggggctacatcgagagggtgagggcagccacgtccgagttggagcacagagttgagcgcacacagaaaaacgtgaaggtgatccagcagaccatgaggggctgggccaggtgcgtgctacctcccaggagagagcacagacgagaggcagccttcaccttggaggacaagggtgatttgtttacaaaaaaatacaagttaatccaaggagatggctgcaagatccacaacttggtcgaggaaaataggaagctcttcaaagccaatccctctctggatacctggaaaatttatgtagaattcattgacgacattgtggtggaaggcttttttcaggctataatgcacgacttagacttctttctgaagaatacagagaaacaattgaaaccggcaccgttttttcaagcacaaatgatcttgttgcctcctgagattgtgtttaaaccttccctagacagagaggctggggatggcttctatgatcttgtagaagaaatgttatgcaatagttttagaatgtctgcccagatgaaccgaatagcaacacacctggaaattaaaaattatcagaatgatatggataacatgttaggcctggcagaggtcaggcaggagatcatgaacagagtggtgaatgtcatcaacaaagtcttagatttcagaaacaccctggagacccacacttacctctgggtggatgatcgagctgagtttatgaagcattttctcttgtatggccatgctgtgtcttccgatgaaatggatgctcatgcaaatgaagaaattcccgaacaaccaccaactcttgagcaattcaaagaacagattgacatttatgaagctttgtatgttcaaatgagcaaatttgaggactttagagtgtttgatagttggttcaaggtggacatgaagcctttcaaagtgagcttgttaaccataattaagaaatggagctggatgtttcaggagcatcttttgagatttgtcattgacagtctgaatgagctacaagaatttataaaggagacagattccggacttcagagagaattaaatgaaggtgatcatgatggtttagttgacatcatggtgcatcttctggctgtaagaagccgacagagagctactgatgaactctttgaacctctaaaagaaacgatcaccctcttggaaagctatggccagaagatgcctgagcaggtctatattcagctagaggaattacctgaaagatgggaaactaccaaaaagatcgcagcaactgtcagacatgaagtctcacctctccataatgcggaagtcactcttataaggaaaaaatgtattttgtttgacgcaaagcaggcagagttcagagagagattcagacactatgcccctcttggatttaatgcagaaaatccatacacagcgcttgataaggcaaatgaagagcttgaggccttagaagaagaaatgttgcagatgcaagaatctactcgtctttttgaagtggctcttccagagtacaaacaaatgaaacagtgtcgcaaagaaataaaattgctcaagggactgtgggatgtcattatttatgttcgaagaagcattgataattggactaaaacccagtggagacagattcatgtggaacagatggatgtagaactcagaaggtttgccaaggaaatttggtcactcaacaaggaagtccgcgtctgggatgcttacacgggcctggaaggcacagttaaggacatgacagcctccctgagggccatcacagagttacagagccctgccctcagggacaggcattggcaccagctgatgaaagctattggggtcaagtttttaataaatgaagccacaactttggcagatttgttagcactgcggttacacagagtggaagatgatgtccgaaggattgtggacaaggcggtgaaagagctggggactgagaaggttattactgaaatcagtcagacctgggcaaccatgaagttttcttacgaagttcactatcgaacaggcattccattactaaagtctgatgaacaactttttgaaactctagagcacaaccaagttcagttgcagactcttcttcaaagcaagtatgtagaatatttcattgagcaagtgttaagctggcaaaataaattaaacatagcagacttggtcatcttcacttggatggaagtccagcgaacttggtctcacctggaaagcatttttgtctgttcagaagatattcgaatccagcttgtgaaagatgctagaagatttgatggggtggatgctgaatttaaggagttaatgttcaagacagccaaagtagaaaatgtgttagaagcaacgtgcagacctaatctctatgaaaaacttaaagatttacagtccaggctttctctttgtgaaaaagctctcgctgaatacctggaaaccaagcgcatagcctttcctcgcttctatttcgtctcttctgctgatttacttgacattctctcaaaaggagctcagcctaaacaggtaacatgtcaccttgccaaacttttcgacagcattgcagatctgcagtttgaagacaatcaggatgtttctgcacacagggcagttggaatgtacagcaaagaaaaggagtatgtcccattccaagccgagtgtgaatgtgtgggccatgtggaaacatggcttctgcaacttgaacagactatgcaagaaacggtgcgtcattctataacagaagccatagtggcctacgaggaaaaacctagggaactgtggatttttgatttcccagctcaggttgcactaaccagctcacaaatatggtggaccacagatgtaggaatagccttcagtagactggaagaaggctacgaaacagccctgaaggatttccataaaaaacagatttctcagctgaatacactgattacacttttgctgggagaacttccacctggagacagacagaagatcatgacaatttgtaccatagatgtccatgccagagacgtggtggcaaaacttatttctcagaaggttgtcagtccccaagcttttacatggctgtctcaacttcgtcaccgatgggaggatacccagaaacactgctttgttaatatttgtgatgcccagttccagtacttctatgaatacttaggaaacagccctcgactagtgatcactcctctaactgacaggtgttatattaccttaactcaatcacttcatctaaccatgagtggggctcctgctggcccagctggtaccgggaaaacagagaccaccaaagacctaggacgtgcccttggcatgatggtctatgtattcaactgttcagagcaaatggactacaaatccataggcaatatctataagggattggtgcagacaggagcttggggctgctttgatgagttcaaccgaatctctgtggaagttctgtcagtggtggcagtacaagtgaaaatgattcatgatgccatcagaaacaggaagaagagatttgtatttcttggggaagctatcacactgaagccatcagttggaatatttattactatgaacccgggttatgctggtcgaaccgaattaccggaaaatctcaaagctcttttcagaccctgtgccatggtggcccctgacattgagctaatctgtgaaatcttgttagttgctgaaggttttgtggatgcgcgtgcattagcccgaaagttcattacgttgtacacgctttgcaaggagcttctctccaagcaggatcattacgactggggacttcgtgctattaagtctgtcttggttgtggctggatctctgaaacgaggagataaaaatagacccgaagatcaggtactcatgagagcattaagggatttcaatatgcccaaaatagtgactgacgacatcccagtgtttctgggcctggtcggtgacctgtttccagccctggatgtgccccggaggaggaagctgcactttgaacagatggtcaggcagtctaccctggagctccgcctgcagcctgaagagagcttcatcctcaaagttgtccagcttgaggaactgttggctgtgcggcactcggtctttgtagttggaaatgcaggcacaggaaagagtaagattttgagaacactgaaccgaacatatgttaacatgaaacagaagccggtttggaatgacttaaaccctaaagctgtgacaacagatgaactctttggtttcatacatcatgctacccgagaatggaaagatggcaagattgtttactcttattttataggtctcttctcatccattctacgagaacaagcaaatcttaagcatgatggaccaaaatggatagtcctggatggcgatattgaccccatgtggattgaatcactgaatactgtaatggatgataacaaggtgctgaccctcgccagcaatgagcgcattgcactcactcccttcatgaggcttctgtttgagatacatcacttaaggagcgcaaccccggccactgtttccagagctggtattctgtatgtgaacccacaagatctgggctggaatccgtatgtggccagttggatagacagaaggcggcatcaatcagaaaaggccaatttgactattctttttgataaatatgtccctgcatgcttggataaactgagaacaagctttaaaaccatcacttcaattcctgagagtagcctggtgcagactctatgtgttcttttggagtgcttgctgactcctgaaaatgtaccttctgacagcccaaaagaagtttatgaagtctattttgtatttgcttgtatctgggcttttggaggcaccctgctacaagatcagatttctgattatcaagctgacttcagtcggtggtggcagaaagagatgaaagcagtgaaatttccgtcgcagggaacaatctttgattattatgtggaccacaaaactaagaaattattgccctgggctgacaaaattgcccagtttactatggatccagatgtgcctctgcagacagttctcgttcacacaacagagacagctcgtcttagatatttcatggagttgttgcttgagaaaggaaaacctctaatgctagtaggaaatgcaggagtgggaaaaacagtctttgtaggtgacacattggcaagtctctctgaggattacatagtatcccgtgtgcctttcaactactacacgacatccacagctctgcaaaaaattcttgagaaacccctagagaaaaaagctggtcataactatggtcctggaggaaataaaaaattgatttattttatcgacgacatgaacatgcctgaagtggacttatatggcaccgttcagcctcacaccctgatccggcagcatattgattatggacattggtatgatagacagaaggtgatgcttaaagaaatccataactgccagtatgtcgcctgcatgaatccgatggtgggcagcttcaccatcaatcccaggctacagagacatttcacagtgtttgcattcaattttccatctttggatgcactaaacaccatctatggccaaatctttagcttccatttccaacagcaagcatttgctccatcaattctcaggagtggccccactttgatccaggcaacaatagcattccatcagacaatgatgtgtaactttttacccacggctattaaattccactacatctttaatctgagagatttatcaaacgtcttccaggggattttatttgcttctcctgagtgtttaaaaggtccacttgatttaatacatctgtggcttcatgaatctgcccgtgtttatggagacaaactgatagacaaaaaagattgtgatttgtttcagagaagaatgctggaaactgcttataaatattttgaaggtatagatagtcacatgctgcttcaacagcccctcatttattgccactttgctgatagagggaaggacccacattacatgccagtgaaggactgggaagtgctgaagacgattcttacagaaacgttagacaactacaatgaactaaatgctgccatgcacctagttttgtttgaagatgccatgcaacatgtgtgtcgcatcagccggatcttacgaacccctcagggctgtgctctcttggttggagttgggggcagtggcaagcagagcttgtccaggctggcagcttaccttcgtggccttgaggtctttcagatcactctgaccgagggctatggaatccaggaacttcgggtagatcttgccaatttgtacatccgaactggagccaagaacatgcccactgtgttcctgctgacagatgcccaggttctagatgagagcttcctcgtgctgattaatgacttgctggcatcaggagaaatcccagatctgttcagcgatgaagatgtggacaagataatttctggaattcataatgaagttcatgctctgggcatggtagactccagggaaaactgttggaaattctttatggccagggtgcgactacagctcaaaatcattttgtgtttctctccagttggtcgcacgctgagagttagagctcggaagttcccagccatagttaactgcacggctattgactggtttcatgcgtggccgcaggaggctctggtctccgtcagcaggaggttcattgaggaaaccaagggaattgagccagtgcacaaagactctattagccttttcatggcacatgttcacaccactgtaaatgaaatgagtaccagatattaccagaatgagagaagacacaactataccaccccaaagagttttctagaacaaatatcactgtttaagaacctgttgaagaagaagcaaaatgaggtatccgagaaaaaagaacgcctggtgaacggcatccaaaagctaaaaaccacagcctctcaggtgggagatctaaaagccagacttgcctctcaagaagccgagctgcaactgagaaatcatgatgccgaagctctgatcacaaagatcggccttcagacggagaaagtgagccgggaaaagaccatcgctgatgctgaggagcgaaaggtgacagccattcagactgaagtgttccagaaacagagagaatgtgaagctgacttactcaaggctgagcctgcacttgtggctgctacagctgcactcaatacactcaacagggtcaacctcagtgagctgaaagcctttcccaaccctcccatcgcagttaccaatgttactgcagccgtgatggtccttctggctcctcggggaagagtgcccaaagaccgaagttggaaagcagctaaagtcttcatgggaaaggttgatgattttttgcaagcattaattaactatgacaaagagcacattccagagaactgtctaaaagtggtgaatgaacactatttgaaagacccagagtttaatccaaacctgattcgaaccaaatcttttgcagcagctggcctgtgtgcctgggtcatcaacatcattaaattctatgaggtctactgtgatgtggagccaaaacgccaagcattagcccaagcaaacttagaactggctgcagctactgaaaaactagaggctatcaggaaaaagcttgtggatctggatcgaaatctgagcagactcacggcttcatttgaaaaagcaacagctgagaaagtccggtgtcaagaagaggtgaaccaaaccaacaaaaccatcaaattagctaacagacttgtcaaggaacttgaggcaaagaagattcgctggggtcaatccattaagtcctttgaagctcaagagaagacactctgtggagatgttcttctcacggcggcatttgtgtcttacgtcggacccttcacaaggcagtatcgccaggagctggtgcactgcaagtgggttccctttcttcaacagaaggtttccattccactaaccgaaggcctggacttgatatccatgttgacggatgatgctacaattgccgcctggaataacgaaggactgcccagtgacagaatgtccaccgaaaatgccgctatcctaacacactgtgagcgctggcctctggtgatagatccccagcaacagggaattaagtggatcaagaataagtatggaatggacctgaaagtcacacatttgggccagaaagggtttttgaatgccattgaaactgctttggcctttggtgatgtcatcttaattgaaaatctcgaggaaacgatagatccagtcctggatccactacttggcaggaacacaattaaaaaaggaaagtatatcaggattggagataaagaatgtgaatttaacaagaactttcgccttatccttcacacaaaattggcaaatcctcactataagccggaattacaagctcagacaactctcctcaatttcacagtcacagaagatggtctagaagcccagctgctggcagaggttgtcagtattgaaaggccagatttggagaaacttaagttggtattgacaaagcaccaaaatgattttaaaattgagctcaagtatctggaagacgatctccttttgcgcctttctgcggcagagggaagctttctggatgacaccaaactggtagagagattggaggcaacaaagaccaccgtggcagagatagagcacaaggtgattgaagccaaagaaaatgaaagaaaaatcaacgaggcccgagaatgttacagaccagtggcagcaagagcatctcttctttattttgttattaatgacctccaaaaaatcaaccccctctaccaattctctttgaaggcttttaacgtgctgttccacagagcgatcgagcaggctgacaaggtggaagacatgcagggacgcatctctatcctgatggagagcatcacccatgctgtcttcctctacaccagccaggcgctgtttgagaaggacaagctcaccttcctgtcccagatggcttttcagattttgttgagaaagaaagagatagaccctcttgaattggatttcctgcttcgattcacagttgaacacactcatctgagtcccgttgacttcctaacttctcagtcatggagtgctatcaaggcaattgccgtcatggaagaatttcgaggcatagaccgagatgtggaaggatctgccaagcagtggaggaagtgggtagaatccgagtgtccagaaaaagaaaaattacctcaagaatggaagaagaaaagtttaatacagaagctgattcttctgagagcaatgcgccctgacagaatgacgtatgctctcagaaattttgtagaggaaaaactgggtgcgaagtatgtggagaggaccagattggacttagttaaagcattcgaagaaagcagcccagccacccccatattcttcatcctgtctccgggggtagatgcccttaaagacctggagattcttggcaaaagacttggctttacaattgactctggaaaattccacaatgtgtctttaggacaaggtcaggagacggtggcagaagtggccctggagaaagcttccaaaggaggacactgggtcatcctccaaaatgttcatttggtagccaagtggctaggaaccttggagaagctccttgaaagattcagccaaggaagccacagagattacagggttttcatgagtgctgagtctgcacctacaccagatgagcatatcatccctcaaggactcctggaaaattccattaagatcactaatgaacccccaacagggatgctggccaatttgcatgccgccctgtacaactttgatcaggatacacttgaaatatgctccaaggagcaggagtttaaaagcatccttttttctctctgctacttccacgcctgtgttgctgggagactgaggtttggcccccagggctggagccgaagctatccttttaatcctggagacctcaccatttgtgccagtgtcctctacaactacttagaggcaaactctaaagtcccatgggaagatctccgttatctctttggtgagatcatgtatggaggccacatcacagatgactgggatcgcaaactgtgtcgggtgtatttagaagaattcatgaatccatctctgactgaagatgaactgatgctggcaccaggttttgctgccccaccctacctagattatgcaggctaccaccagtacatagaggagatgcttcctccagaaagcccggcactgtatggcctccacccaaatgctgaaatagaattcctgacagtgacatccaacactctcttcagaactttgctggagatgcagcccaggaatgcactcagtggtgatgaactggggcagtctacagaagaaaaggttaagaatgtcttggatgacattttggagaaacttccagaagagttcaacatggcagagataatgcaaaaaaattcaaatagaagcccatatgttcttgtttgcttccaagaatgtgagaggatgaatattctcattcgggaaatacgtatatcacttgaacaactggaccttagtttgaagggggaattggcattatctcctgctgtggaagcccagcagtttgcattgagttatgacacggtaccagacacttggagcaaactggcttatccttctacttatggcctagcccagtggttcaatgacctcctcctgcgatgccgagaactcgatacttggacacaagaccttacccttccggctgtcgtgtggctctccggcttcttcaaccctcagtccttcttaactgcaatcatgcagacgatggctcgaaaaaatgagtggcccctggataaaacgcgcttgactgctgatgttaccaaaaaaacaaaggaagattatggacaccccccaagggaaggtgcatacctccacggactcttcatggagggcgcccgctgggacacccaagcaggaaccattgttgaagcccgtctcaaggagctggcatgccctatgccggtcatctttgcaaaagccacccccgtggacagacaagaaaccaaacagacctacgagtgccctgtgtatagaaccaaactgagaggccccagctacatctggaccttcaggctgaagagcgaagagaagactgcaaaatgggttctggctggagtggctctgcttctagaagcgtaaggtaacactggcattcctctagcctctgctggagtgcagtgaggattttctagcatgttgctgcactgttcccatgcacattattctaactttttagtaactcacacgtgcattcttttttcaacgctatccttagagtgaaagtcagaaaaaaatactagaaactaactcagggctgagcgtggtggcacacgactgtaatcccagttactcaggaggtaggagaatcacttgaacctaggaggcaaaggttgcagtgagccgaggttgcaccactgcactccctcctgggcaacagaacaagactccatctcaaaaaaaaaaaagtacatcataaaagtacatcatatgtgaacatgcaaaagcaatgcagccggaaagaacggagattttaatttttaacaaacaacaaattaaattattagcccttaaactctttcaaaatataaaagcagcaggccccaggtgagtcctgaaggaagaggctagcactctgtaaggcctccagtgtccagtgtctacaatgttgatggtccccttttgttcagtcaagttttaataaaaataaaactgttctacagttaa

SEQ ID NO: 2 - protein, 4516 aa, Homo sapiens
MAAQVAAREARDFREAPTLRLTSGAGLEAVGAVELEEEEENEEEAAARRARSFAQDARVRFLGGRLAMMLGFTEEKWSQYLESEDNRQVLGEFLESTSPACLVFSFAASGRLAASQEIPRDANHKLVFISKKITESIGVNDFSQVVLFGELPALSLGHVSAFLDEILVPVLSNKNNHKSWSCFTSQDMEYHIEVMKKKMYIFRGKMSRRTLLPIPTVAGKMDLDQNCSENKPPSNERIILHAIESVVIEWSHQIQEIIERDSVQRLLNGLHLSPQAELDFWMMRRENLSCIYDQLQAPVVLKMVKILTTKQSSYFPTLKDIFLAVENALLEAQDVELYLRPLRRHIQCLQETEFPQTRILIAPLFHTICLIWSHSKFYNTPARVIVLLQEFCNLFINQATAYLSPEDLLRGEIEESLEKVQVAVNILKTFKNSFFNYRKKLASYFMGRKLRPWDFQSHLVFCRFDKFLDRLIKIEDIFATTLEFEKLERLEFGGTKGAILNGQVHEMSEELMELCKLFKQSTYDPSDCTNMEFESDYVAFKSKTLEFDRRLGTIICEAFFNCNGLEAAFKLLTIFGNFLEKPVVMEIFSLHYSTLVHMFNTELDVCKQLYNEHMKQIECGHVVLNKNMPFTSGNMKWAQQVLQRLQMFWSNFASLRYLFLGNPDHALVYQKYVEMTTLLDQFESRIYNEWKSNVDEICEFNLNQPLVKFSAINGLLCVNFDPKLVAVLREVKYLLMLKKQDIPDSALAIFKKRNTILKYIGNLDLLVQGYNKLKQTLLEVEYPLIEDELRAIDEQLTAATTWLTWQDDCWGYIERVRAATSELEHRVERTQKNVKVIQQTMRGWARCVLPPRREHRREAAFTLEDKGDLFTKKYKLIQGDGCKIHNLVEENRKLFKANPSLDTWKIYVEFIDDIVVEGFFQAIMHDLDFFLKNTEKQLKPAPFFQAQMILLPPEIVFKPSLDREAGDGFYDLVEEMLCNSFRMSAQMNRIATHLEIKNYQNDMDNMLGLAEVRQEIMNRVVNVINKVLDFRNTLETHTYLWVDDRAEFMKHFLLYGHAVSSDEMDAHANEEIPEQPPTLEQFKEQIDIYEALYVQMSKFEDFRVFDSWFKVDMKPFKVSLLTIIKKWSWMFQEHLLRFVIDSLNELQEFIKETDSGLQRELNEGDHDGLVDIMVHLLAVRSRQRATDELFEPLKETITLLESYGQKMPEQVYIQLEELPERWETTKKIAATVRHEVSPLHNAEVTLIRKKCILFDAKQAEFRERFRHYAPLGFNAENPYTALDKANEELEALEEEMLQMQESTRLFEVALPEYKQMKQCRKEIKLLKGLWDVIIYVRRSIDNWTKTQWRQIHVEQMDVELRRFAKEIWSLNKEVRVWDAYTGLEGTVKDMTASLRAITELQSPALRDRHWHQLMKAIGVKFLINEATTLADLLALRLHRVEDDVRRIVDKAVKELGTEKVITEISQTWATMKFSYEVHYRTGIPLLKSDEQLFETLEHNQVQLQTLLQSKYVEYFIEQVLSWQNKLNIADLVIFTWMEVQRTWSHLESIFVCSEDIRIQLVKDARRFDGVDAEFKELMFKTAKVENVLEATCRPNLYEKLKDLQSRLSLCEKALAEYLETKRIAFPRFYFVSSADLLDILSKGAQPKQVTCHLAKLFDSIADLQFEDNQDVSAHRAVGMYSKEKEYVPFQAECECVGHVETWLLQLEQTMQETVRHSITEAIVAYEEKPRELWIFDFPAQVALTSSQIWWTTDVGIAFSRLEEGYETALKDFHKKQISQLNTLITLLLGELPPGDRQKIMTICTIDVHARDVVAKLISQKVVSPQAFTWLSQLRHRWEDTQKHCFVNICDAQFQYFYEYLGNSPRLVITPLTDRCYITLTQSLHLTMSGAPAGPAGTGKTETTKDLGRALGMMVYVFNCSEQMDYKSIGNIYKGLVQTGAWGCFDEFNRISVEVLSVVAVQVKMIHDAIRNRKKRFVFLGEAITLKPSVGIFITMNPGYAGRTELPENLKALFRPCAMVAPDIELICEILLVAEGFVDARALARKFITLYTLCKELLSKQDHYDWGLRAIKSVLVVAGSLKRGDKNRPEDQVLMRALRDFNMPKIVTDDIPVFLGLVGDLFPALDVPRRRKLHFEQMVRQSTLELRLQPEESFILKVVQLEELLAVRHSVFVVGNAGTGKSKILRTLNRTYVNMKQKPVWNDLNPKAVTTDELFGFIHHATREWKDGKIVYSYFIGLFSSILREQANLKHDGPKWIVLDGDIDPMWIESLNTVMDDNKVLTLASNERIALTPFMRLLFEIHHLRSATPATVSRAGILYVNPQDLGWNPYVASWIDRRRHQSEKANLTILFDKYVPACLDKLRTSFKTITSIPESSLVQTLCVLLECLLTPENVPSDSPKEVYEVYFVFACIWAFGGTLLQDQISDYQADFSRWWQKEMKAVKFPSQGTIFDYYVDHKTKKLLPWADKIAQFTMDPDVPLQTVLVHTTETARLRYFMELLLEKGKPLMLVGNAGVGKTVFVGDTLASLSEDYIVSRVPFNYYTTSTALQKILEKPLEKKAGHNYGPGGNKKLIYFIDDMNMPEVDLYGTVQPHTLIRQHIDYGHWYDRQKVMLKEIHNCQYVACMNPMVGSFTINPRLQRHFTVFAFNFPSLDALNTIYGQIFSFHFQQQAFAPSILRSGPTLIQATIAFHQTMMCNFLPTAIKFHYIFNLRDLSNVFQGILFASPECLKGPLDLIHLWLHESARVYGDKLIDKKDCDLFQRRMLETAYKYFEGIDSHMLLQQPLIYCHFADRGKDPHYMPVKDWEVLKTILTETLDNYNELNAAMHLVLFEDAMQHVCRISRILRTPQGCALLVGVGGSGKQSLSRLAAYLRGLEVFQITLTEGYGIQELRVDLANLYIRTGAKNMPTVFLLTDAQVLDESFLVLINDLLASGEIPDLFSDEDVDKIISGIHNEVHALGMVDSRENCWKFFMARVRLQLKIILCFSPVGRTLRVRARKFPAIVNCTAIDWFHAWPQEALVSVSRRFIEETKGIEPVHKDSISLFMAHVHTTVNEMSTRYYQNERRHNYTTPKSFLEQISLFKNLLKKKQNEVSEKKERLVNGIQKLKTTASQVGDLKARLASQEAELQLRNHDAEALITKIGLQTEKVSREKTIADAEERKVTAIQTEVFQKQRECEADLLKAEPALVAATAALNTLNRVNLSELKAFPNPPIAVTNVTAAVMVLLAPRGRVPKDRSWKAAKVFMGKVDDFLQALINYDKEHIPENCLKVVNEHYLKDPEFNPNLIRTKSFAAAGLCAWVINIIKFYEVYCDVEPKRQALAQANLELAAATEKLEAIRKKLVDLDRNLSRLTASFEKATAEKVRCQEEVNQTNKTIKLANRLVKELEAKKIRWGQSIKSFEAQEKTLCGDVLLTAAFVSYVGPFTRQYRQELVHCKWVPFLQQKVSIPLTEGLDLISMLTDDATIAAWNNEGLPSDRMSTENAAILTHCERWPLVIDPQQQGIKWIKNKYGMDLKVTHLGQKGFLNAIETALAFGDVILIENLEETIDPVLDPLLGRNTIKKGKYIRIGDKECEFNKNFRLILHTKLANPHYKPELQAQTTLLNFTVTEDGLEAQLLAEVVSIERPDLEKLKLVLTKHQNDFKIELKYLEDDLLLRLSAAEGSFLDDTKLVERLEATKTTVAEIEHKVIEAKENERKINEARECYRPVAARASLLYFVINDLQKINPLYQFSLKAFNVLFHRAIEQADKVEDMQGRISILMESITHAVFLYTSQALFEKDKLTFLSQMAFQILLRKKEIDPLELDFLLRFTVEHTHLSPVDFLTSQSWSAIKAIAVMEEFRGIDRDVEGSAKQWRKWVESECPEKEKLPQEWKKKSLIQKLILLRAMRPDRMTYALRNFVEEKLGAKYVERTRLDLVKAFEESSPATPIFFILSPGVDALKDLEILGKRLGFTIDSGKFHNVSLGQGQETVAEVALEKASKGGHWVILQNVHLVAKWLGTLEKLLERFSQGSHRDYRVFMSAESAPTPDEHIIPQGLLENSIKITNEPPTGMLANLHAALYNFDQDTLEICSKEQEFKSILFSLCYFHACVAGRLRFGPQGWSRSYPFNPGDLTICASVLYNYLEANSKVPWEDLRYLFGEIMYGGHITDDWDRKLCRVYLEEFMNPSLTEDELMLAPGFAAPPYLDYAGYHQYIEEMLPPESPALYGLHPNAEIEFLTVTSNTLFRTLLEMQPRNALSGDELGQSTEEKVKNVLDDILEKLPEEFNMAEIMQKNSNRSPYVLVCFQECERMNILIREIRISLEQLDLSLKGELALSPAVEAQQFALSYDTVPDTWSKLAYPSTYGLAQWFNDLLLRCRELDTWTQDLTLPAVVWLSGFFNPQSFLTAIMQTMARKNEWPLDKTRLTADVTKKTKEDYGHPPREGAYLHGLFMEGARWDTQAGTIVEARLKELACPMPVIFAKATPVDRQETKQTYECPVYRTKLRGPSYIWTFRLKSEEKTAKWVLAGVALLLEA

SEQ ID NO: 3 - DNA, 162 bp, Homo sapiens
gtttgtgatttttgttaaaaaaaagatactagggcctattatgaatataaatattttgaattttaattattatatagatctatatgatatttttacacctttaagacagagaaaatagctaacatatttggcactttttgttttggggttttctttgctcag

SEQ ID NO: 4 - DNA, 1322 bp, Homo sapiens
gtcagtatccttggtctcactaatgaacctttttatgactgtgaagtgttacttcttggtttgcatcatctgagatgaagtgtaatttatgaaaagacccccctcagcatagcctctacttgctgtgttatgcctttttgctgtctagaaaaagtcgcctgatccgaccaacaatttagaaaaaggcaccctcctccaggctgtccccaggacagcagagcggaggctggcttccatctccatttgtcatctgggctggtgtccacgttcagtacacagattgcacaactaatcagggtggccctattactcatctctcctcattctttctcaaggacagacctagctccctggctcccagcaactctggagtgaggccaaagttgacacagagatagtctggcaaactcatctagctttctcaggactttccagattttagcactgaaagtatcacatccctgaaatccacttgctagaaacccctcagtccggagcaaatcagggcatttggtcctcttatgcagaggcgtggatggtgctaaggaatgttagagggtagggaggggcagagggtggcagcttccaagaagcagattctggatattcttagcaaaagccggaaagaaaatggctttcctgcaacccatatggctggctcaactcttcacttacaggagacatgagtgagtttgggtatgtcgaagagctgaggatgtctcaaggacccaatataggggagggaaggagagtcaatgtgagagaaggggagtcattgtcacaagacaatgtggaaatgagaattatttttcacatggtaaatatgaataatgacactgtaagactattttttttgccaatgaactgagattaaattagtgataaaactaggttaaatcacagaagcagctgggaattatgttcagtgaatagggttctttaggaagaaaagacaaatgagccactacctgattcctgggatttcaaccaaattataataaatgaacaatgagattctctttaaaaatgggttttgtgggcaaaggagataagaaacgtgccgacaagggaaagatgtgaaacaaagaaaatagtgtagcacggatgtgactaaactgtggcccttgcttccggcaggtgacgcctcaggaattgtgagttaaaaaggagtgtcttagattctccacgtatttcaagacgagagatgtactcagcacctgacttgagtatattttagttaagattcatttcaccagcctttaggcaaaaggaaccgttcatatatgtggaatataaagtctaactttttttcccccaatcag

SEQ ID NO: 5 - DNA, 1515 bp, Homo sapiens
gtttgtcaaagacaggctgtatgctattctagcaaagttttgtaaagtaacatggttttgagcatcgtatttataaaaatgaatgcatttttgcatgagtaagcaggagtcaggagacttttcctgcaaagggccaggtaataaatatttcaggctgtgtggatcatgcagcctgtctaacaccttctgaatgctgccattacaacatgaaagaagacatgggtaataaataaacaaatggatatggttatatgccaataaagctttacttgtagacactgaaatttgaatttcacatagttatcatgtgtcacaaaatacactgttttttatttttttcactgatttaaaaaggtaggctaggctcggtggctcacacctataatcccagccttttgggagtccaaggtaggaggatgacttgaggccaggagttcgagacaagcctaggcagtatggtgagatcccttctctactaaaaaatttttaataattggctaggcaaggtggcccaagcttgcagtcctagctacttgggaggctgaggtgggaggatcacttgagccaaggtagtttgaggttgcagtgagctatgactgcagcactgcactccagcctgggtgacagaatgagaccctgtctccaaaataaatttaaaaaccatttttagctgctaggtcgtgcaaaaacaaacatcaggccatatttgtcccatgcaccatagattgccgacctttgtggtaataaaggtaaacagattcatcttcagaagtaaaagactgagaaaggacaacaggtagaaaataacactactagtttcggtttttcttttctgtaaaagccatagaaagcacgtggcatggtgcatgtaatgataattgattggagagatttctagcaaaggagagtgactatttctcataaaaattgagattaaaaggttttttctttctacctgaagtagaagtcacagataaattttaagtcatttaacttttgaggaagaaaaaatagattgtttatatctctctcatttttatatgtaggtatgctttttaagattctgggcgctctctcattctctgtctctctctttctctctctcccttcctctctttctccctttctctctccttctcctccccctctgcccccacctcctcctacttgtctccctttctcttctttttcctcttcccattctcccttccttgtctccttttgcctttattctttttcgtttgatttagaagagaaaaattctgtgtgggcctaaagtggtgacagtgcttatcacatttatttttataaaaacggtgtgcttctacattgcttttgatcttcatctaagagttgaaaagcaaaagtaaacagatgtggtgtcagacattcttagaagtatctttgaccttgcctcttcattctttgttcttcatttttttaattcttgaaatgattaaaagtttagaaaagattgttctaatatccacggccccgtattgtactttcatgcag

SEQ ID NO: 6 - DNA, 126 bp, Homo sapiens
gtaagttagtaagagaataatgtgtaaaactttattctctaacattattcctgattgggaattattcaatataatagcgtaaaaaccccttctgttaaattctgagtgcctcactttatcatttag

SEQ ID NO: 7 - DNA, 4954 bp, Homo sapiens
gtatgtttagaaatagtttacaggaccagtttccagttttgtgtgggacagggtcatggggaggttaaaacatgtgatctgtacctttttgttatgttatagatctatactgcttgccctgttctctcctaagtctatgcattctctgggtagtcccatccctgatggttctaaccccagctgacaatgccaaatccataccctccagcctggctgtgtcttctgaactgaaagttctgtgtttctggctgctctggggcctcgccttcagcatgctgttctacaagaagccccactcagtgtgcctgagacagatctcctcctctgccccatactccaacttgcccctgctcttccttcttttcttttgctcagtggtacattcttcccggtagctccagttaaaaacgtggaagacatcactcttcctgatttctgtgtcctgtgatctcagaccctttcagtttcacctgtgaaatagctgccaaccctattccctcttctccatccctgcagcctaaattcaggagcaaagcatctttagattgctctggttatcatagcgtcttagtagctcagatcgccataacaaaataccacaaattgggtgacttaagcaatagaaatttatttctcacagttctggggaccaaaagtccaagattatggtgtcagcatggttgagttccagcaaggactctcttcccacctttcagaccgccactttcccgctgtgtccccatgtgacagagagagaaagacatactggtttctcttttaatgaggacactaatcccatcatgaggaccctgtattcatgacatcatctaaatctaattatgtcccaaggccccaccaccaaatatcatcacactgtggggtagggcttcaaaatatggatttgggggtgggatgtgggaggggggatcacaattcggtttgtagcatttgataatcttcaaattggtctccctctctccagtccctcttacttctaaccagtctcacacactattcccaggatagtcatttgtgaaccatagtcaaccctgggtcaattcctttttcactgatcttttttttagctgaaagtgaaatattcatgcctgtacagattgcttgcatagcccttttgtgtcttctgatttcctgtgggaaaacacttgagacaaacatttgcaccagcacacagatcccacgaactgcttagacttaccaaagtgcaaagtgggaattattcaactttagaaatttgcagatctaattgccaccatgcacctctatgtgtaatttttctcatgtgatctttacaatggttcttcaaggtatcctcattgcacaaataaagtaaagctcagaggggcaaagtaacttgcccaaagtcacgctgatgggaaatggcaacatcaggattcccacccaagtgtgtctttctccaaaagccacaccttccatggtacctcagggcctctcatggttcatagtgcttctatgggtacccctgaatttttacattaaaaatagatagcacacattttccaggcttccttgctctttccaggtgtaaatggggcatttacattgaaaggcatcgcagccaagagtgacctatatttctgttggtttgttcactgtagatcagaaaataaatggttgatataatctgttggccagttccacttctcgccactaggttaccataactcatgcctattattataataatatagttggaattcacttggcctcctagatcttatttcagccattctcagaagccagattccttctgtaatttccaagtgctttctaaagattagcagccaatactgtgtgagagtcaagtgttcgtgtcagggacttcatgtggctttctcagaggcacggtttacaacatgtgtgggtttcttgttaatggaggccacatgcagacagaaggcaaccctccttctccaccaggtatttctttcaatatttacccttttctccctttctgggactgtaaaatcaggtgggatgttgatagcggacatcttccccctcctgctcataacccctcacctccctcccagctgaccctcttttaatctttccagtttccaaatgtatcagccttaccccctagttatataagctagaagctggatatcactggcaccttcccttcgctaattaatagtcaacatgagcagcaatagcagcttaacattttcaaaagggctctaactacagagcatttaaaacagaggctatgaggttggcaatatgcatgaaacagacacaataaatgttctcaggatagccacttggtagcttaacaaattgagtgtctaatatgtgtcagggaatgtgcttagagcttttcatagggcacctcatttactcttcaaaaactataaaactctatatggattggtacaactcttcttttacaaataaggaactgaagcttagaaaggtattttaatctgcttccaggcacaaagctaacccatgtctaagccaggttcaaacagcagcggactgactcaggacctatgttcttcagccattgcagccaacctccaaaacaagtttcaaatcaactttatctgaactcccataaactgccgtcatttctcacttggcttctgaagtagcctcctcactgggctttctatttctgcatttattctgataattcatcatccactcagcagtcagggtgattttttgaaatgcacgtgaaatcctatgatatactccagagttgaaaacacttgaatggctccttgttgcctttaagattacatttcaaattttaattctatttacaaggcctctgaatctagccctgcttagctctccctatttcttcctctgccacttcccccactcccccactcccccacccctccacccccagctacagggtccagtttcttgagcacaccaagttctttcataccacaggaactttgtacatgctggactcttccttaaacggcacttaaagatgccttccctgaacatccaattaaaataggttgcctctgttattctcacttacagcaaactttactttcaaggcaactgttgatgtctgcgtttgttggcttagcctcagctggacgacacatcttcatgttcctctcctctatgttccccataaactctgtgagaggagagactctgttggttttcttttctactttagaacagatgcgttttagttgaattaattaatgaatgaaagatttgtcaccaaaatcattttgcctatatgtagtggttaggtgaagcaaagtttgatatcaagtaaaagttaaatgtagtctgcctgcagagaatggtgtcacataagtaatatcatctgaactgtcctggctcaagaccaacttttgtagggtgaagaagtatcttctaccaccaatctgaaaataatttaaatgaacctaaagtcataactcatgcatggggtggtagaaggatgctctcagctggtttggaagatggtggggcaagctctccctgcaaacaccgatcacctccttctgcccagtttataggccataatccaatgacatagggacgtcaaagtaaaagcaagtttaaaaacttctatggggaaaaatcacggttgaagtcatagatagaaatgagattgctgaaagcagtagacacacacagtatttttaagagttcatcaaactcactattttaggtcatgctatggatataggagtcactgcgccaggtctcctgtggatataagaggtacagagccaggtcatcgtatgaatataggagtcgctgagccaggtcatcctatgaatataggaaacactgggccaggtcatctgtggatatgggagtaactgggccaggtcatcctatgaatataagagtcactgggccaggtcatcctatgaatataggagtcactgggccaggtcatcctgtggatgcatagaggtatcactggaccaggtcatcatgtggatatggcagtgactggaccagatcatcctagggatataggagtctctgggccagattatcctatggatataggagtcactgggccagatcatcctgtggacatgggcatcactgggacaggtcatcctgtagacatgggcatcactgggccaggtcatcctgtggatatagaagggtttttctagttgttgctcacagttgtccttttctttattttccaaccctaatctagcagtaaatggcttatatctatactgatattaaagcaagcacctcccagtaaagagagcctgttccttgccaatataaatctctctggaggctgaaagttgatccctatgcagttttcagctggaccccttagatttggtctggtgggaaaccaagcccatgcgtttatccattcctccatctctcatggcagggattcccagatttagaattccagagatcaacagagtttcagtaatgatgtgtaggaatggtatagagttgccagttgtttacttttctaagcaagagcatttatgaaaataactatcaccataattttataagggaaaaatattttatcattgaaagggccagagaagctataagctcagaacaaaggacagtatttaaatcaaataatgtcattaatttttaaaaagtacgccattcttatttttcccattttcccataaattgatgtgaacattggtagaagtgaacaccaccgttacaatagtgactacctagcataggaatttgcataatacgttgtcattagattgaataacgaacaggtaatctggcgttcacctaactttaccttgctttttaagttaattatttcacaaaatttgtgcttgatgggtatatggcaattttaggtttctacccctatgatattgttactcataatttgtttctttttttacagttttaattactttgagtctgcaataaggatttcttttgttctccttag